(19) United States
(12) Patent Application Publication
(10) Pub. No.: US 2026/0165684 A1
(43) Pub. Date: Jun. 18, 2026

(54) Determining Venous Congestion with Ultrasound

(71) Applicant: FUJIFILM SONOSITE, INC., Bothell, WA (US)

(72) Inventors: Brittney Klingenberg, Snohomish, WA (US); David Knapp, Bellevue, WA (US); Kristine Kelly, San Diego, CA (US); Shannon Rooney, Toronto (CA); Jae Park, Bothell, WA (US); Sarah Kazemian, Bothell, WA (US); Lillian Larson, Bothell, WA (US)

(21) Appl. No.: 18/983,890

(22) Filed: Dec. 17, 2024

(51) Int. Cl. A61B 8/00; A61B 8/08

(52) U.S. Cl. CPC A61B 8/5223; A61B 8/0891; A61B 8/463; A61B 8/465; A61B 8/467; A61B 8/488; A61B 8/5292; A61B 8/565

(57) ABSTRACT

Ultrasound systems, ultrasound scanners, and methods for determining venous congestion using ultrasound are disclosed. In some embodiments, the ultrasound machine includes a display device configured to display a user interface for the ultrasound machine and a processor. The processor is configured to cause the display device to display an ultrasound image that includes a vein, cause the display device to display a Doppler waveform for the vein, determine a vein type for the vein, determine, based on the vein type, classification waveforms, and cause the user interface to display the classification waveforms.

### FIELD

Embodiments disclosed herein relate to ultrasound systems. More specifically, embodiments disclosed herein are related to determining venous congestion using ultrasound.

### BACKGROUND

Ultrasound systems can generate ultrasound images by transmitting sound waves at frequencies above the audible spectrum into a body, receiving echo signals caused by the sound waves reflecting from internal body parts, and converting the echo signals into electrical signals for image generation. Because they are non-invasive and non-ionizing, ultrasound systems are used ubiquitously. One example in which ultrasound systems are used is the determination of a patient's fluid status, and venous congestion that can develop from fluid overload.

Traditionally, fluid status relied on measurements of the inferior vena cava (IVC) (e.g., a degree of dilation of the IVC). However, IVC measurements can be inaccurate because they do represent a patient's preload in the left ventricle, and they do not quantify the amount of venous congestion from other organs, such as the lungs, liver, gut, and kidneys. Accordingly, more recently researchers have developed a procedure for determining a venous excess ultrasound (VExUS) score based not just on measurements of the IVC, but also the liver, gut and kidneys, as described in Quantifying Systemic Congestion with Point-Of-Care Ultrasound: Development of the Venous Excess Ultrasound Grading System in The Ultrasound Journal 12(1), Article No. 16, 2020, to Beaubien-Souligny, W., Rola, P., Haycock, K., Bouchard, J., Lamarche, Y., Spiegel, R., and Denault, A.

To determine the VExUS score, a user can download a VExUS scorecard that contains example waveforms with varying degrees of abnormalities, and use the example waveforms to determine a degree of abnormality of Doppler waveforms obtained in a pulse wave ultrasound Doppler mode at a hepatic vein, a portal vein, and an intrarenal vein. A downloadable and printable VExUS scorecard is publicly available and can be found in the article cited above. In practice, a clinician (e.g., doctor, nurse, sonographer, operator, etc.) can print the VExUS scorecard or electronically store it on a personal device, such as a smart phone, for referral during an ultrasound examination. However, in this case the clinician is required to look away from the ultrasound system display to reference the contents of the scorecard, and therefore may miss important information on the ultrasound system display. Moreover, a printed scorecard or personal device storing the scorecard is not sanitary and can introduce contagions into the examination room, or facilitate the escape of contagions out of the examination room. Also, the printed scorecard can be easily lost so that it may be unavailable during the examination, and thus cause confusion for the clinician if their expectation includes to rely on the printed scorecard. Further, when not using the scorecard during the examination, the clinician is required to recall its contents from memory, which can lead to misclassifications of waveforms due to poor memory recall. Accordingly, a printed or downloaded VExUS scorecard can introduce undesirable outcomes during an ultrasound examination, and the patient may not receive the best care possible.

### SUMMARY

Ultrasound systems, ultrasound scanners, and methods for determining venous congestion using ultrasound are disclosed. In some embodiments, the ultrasound machine includes a display device configured to display a user interface for the ultrasound machine and a processor. The processor is configured to cause the display device to display an ultrasound image that includes a vein, cause the display device to display a Doppler waveform for the vein, determine a vein type for the vein, determine, based on the vein type, classification waveforms, and cause the user interface to display the classification waveforms.

In some other embodiments, an ultrasound system includes an ultrasound scanner configured to transmit ultrasound at a patient anatomy and receive reflections of the ultrasound from the patient anatomy, the patient anatomy including a vein and a display device. The display device is configured to display an ultrasound image, based on the reflections, that includes the vein and classification waveforms for vein types including a hepatic vein, a portal vein, and an intrarenal vein, the classification waveforms indicating degrees of abnormality. The ultrasound system also includes a processor system configured to cause the ultrasound system to operate in a pulse wave Doppler mode so that the display device displays a Doppler waveform for the vein, determine a vein type for the vein from among the vein types, obtain a selection of one of the classification waveforms for the vein type, and generate, based on the selection of the one of the classification waveforms, a score indicative of venous congestion.

In yet some other embodiments, an ultrasound machine includes a display device configured to display classification waveforms for vein types including a hepatic vein, a portal vein, and an intrarenal vein and a processor system. The processor system is configured to determine a first vein type for a first vein and a second vein type for a second vein, the first vein type and the second vein type determined from among the vein types and being different from one another, generate a first Doppler waveform for the first vein based on first ultrasound generated by the ultrasound machine, and generate a second Doppler waveform for the second vein based on second ultrasound generated by the ultrasound machine. The processor system is also configured to match the first Doppler waveform to a first classification waveform of the classification waveforms for the first vein type, match the second Doppler waveform to a second classification waveform of the classification waveforms for the second vein type, and generate a score indicative of venous congestion based on the first classification waveform and the second classification waveform.

In still some other embodiments, an ultrasound machine includes a display device configured to display a user interface for the ultrasound machine and a processor system. The processor system is configured to cause the display device to display a Doppler waveform for a vein, determine a vein type for the vein, and cause the user interface to display classification waveforms that indicate degrees of abnormality for the Doppler waveform. The processor system is also configured to determine, based on the classification waveforms, a degree of abnormality for the Doppler waveform from among the degrees of abnormality and generate a score indicative of venous congestion based on the degree of abnormality for the Doppler waveform.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The appended drawings illustrate examples and are, therefore, exemplary embodiments and not considered to be limiting in scope.

FIG. 1 illustrates an example ultrasound system in an environment for determining venous congestion with ultrasound during an ultrasound examination in accordance with some embodiments.

FIG. 2 illustrates some embodiments of an example implementation of the ultrasound system from FIG. 1.

FIG. 3 illustrates some embodiments of an example user interface of an ultrasound machine for determining venous congestion with ultrasound.

FIG. 4 illustrates some embodiments of an example user interface of an ultrasound machine for determining venous congestion with ultrasound.

FIG. 5 illustrates some embodiments of an example user interface of an ultrasound machine for determining venous congestion with ultrasound.

FIG. 6 illustrates some embodiments of an example user interface of an ultrasound machine for determining venous congestion with ultrasound.

FIG. 7 illustrates some embodiments of an example user interface of an ultrasound machine for determining venous congestion with ultrasound.

FIG. 8 illustrates some embodiments of an example user interface of an ultrasound machine for determining venous congestion with ultrasound.

FIG. 9 illustrates some embodiments of an example system for determining venous congestion with ultrasound.

FIG. 10 illustrates an example device for determining venous congestion with ultrasound during an ultrasound examination in accordance with some embodiments.

FIG. 11 illustrates an example environment for determining venous congestion with ultrasound in accordance with some embodiments.

FIG. 12 illustrates an example machine-learning architecture used to train a machine-learned model in accordance with some embodiments.

FIG. 13 illustrates an example machine-learned model using a CNN in accordance with some embodiments.

FIG. 14 illustrates an example method for determining venous congestion in accordance with some embodiments.

FIG. 15 illustrates an example method for determining venous congestion in accordance with some embodiments.

FIG. 16 illustrates an example method for determining venous congestion in accordance with some embodiments.

FIG. 17 illustrates an example method for determining venous congestion in accordance with some embodiments.

### DETAILED DESCRIPTION

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

A printed or downloaded VExUS scorecard can introduce undesirable outcomes during an ultrasound examination, and the patient may not receive the best care possible. For instance, a clinician is required to look away from the ultrasound system display to reference the contents of the scorecard, which can distract the clinician and cause them to miss information on the clinical display. Moreover, a printed scorecard or personal device storing the scorecard is not sanitary and can introduce contagions into the examination room, or facilitate the escape of contagions out of the examination room. Also, the printed scorecard can be easily lost so that it may be unavailable during the examination, and thus cause confusion for the clinician if their expectation includes to rely on the printed scorecard. Further, when not using the scorecard during the examination, the clinician may be required to recall its contents from memory, which can lead to misclassifications of waveforms due to poor memory recall.

Accordingly, systems, devices, and methods for determining venous congestion with ultrasound are disclosed herein. In some embodiments, an ultrasound system displays classification waveforms (e.g., example Doppler waveforms that indicate degrees of abnormality) on a clinical display of an ultrasound machine, e.g., simultaneously with an ultrasound image that includes a vein and a Doppler waveform generated for the vein. A clinician can visually compare the Doppler waveform to the classification waveforms to determine a degree of abnormality for the Doppler waveform (and hence for the vein) without taking their eyes off the clinical display. In embodiments, a clinician may select a classification waveform displayed on an ultrasound machine (e.g., with a touch input) to cause the system to generate a score indicative of venous congestion based on the selected classification waveform, and have the system automatically populate a medical worksheet with the score and/or Doppler waveform. In some embodiments, the system can display the classification waveforms as part of a current step of an ultrasound protocol. The system can emphasize the classification waveforms for a vein type of the current step of the ultrasound protocol, such as by increasing the size of the classification waveforms for the vein type compared to classification waveforms for other vein types, or otherwise reducing the visibility of the classification waveforms for other vein types. In some embodiments, the system displays the classification waveforms for only the vein type of the current step of the ultrasound protocol, and does not display the classification waveforms for the other vein types. In aspects of determining venous congestion with ultrasound, the system can automatically adjust a scale for display of the Doppler waveform (e.g., a time scale), based on the vein type and/or current protocol step. These and other aspects of determining venous congestion with ultrasound are described in more detail below.

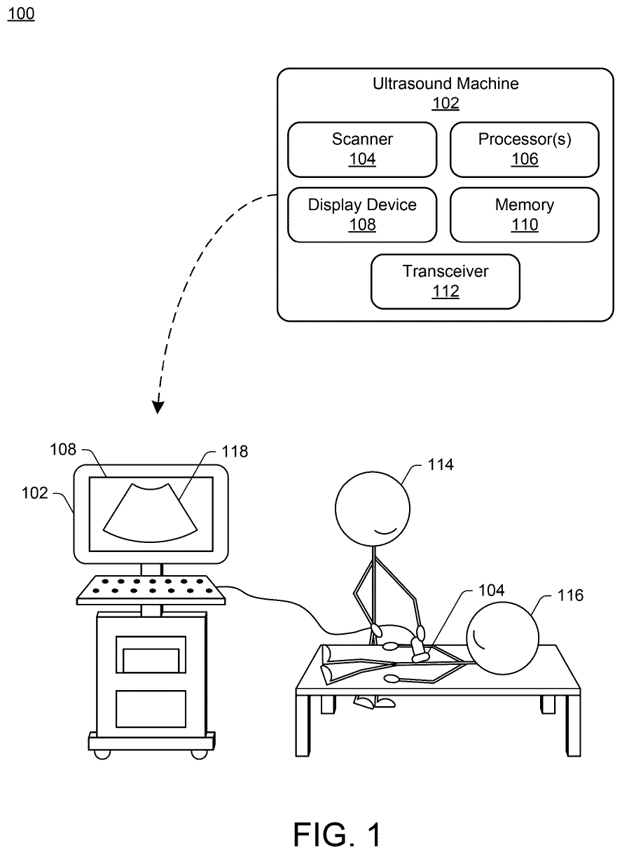

FIG. 1 illustrates an ultrasound system in an environment 100 for determining venous congestion with ultrasound. Venous congestion is used throughout this specification as an example of an ultrasound procedure that can use waveform classification. Other examples of ultrasound procedures that can use waveform classification and benefit from the systems, devices, and procedures disclosed herein include diastology, transcranial Doppler, downstream stenosis, assisted cardiac output, recognition of cardiac waveforms (e.g., aortic insufficiency and cardiac tamponade on mitral valve or tricuspid valve), torsion (e.g., to determine ovary or umbilical cord twist), retinal artery occlusion, etc.

The ultrasound system in FIG. 1 includes an ultrasound machine 102 and an ultrasound scanner 104. The ultrasound machine 102 generates high-frequency sound waves (e.g., ultrasound) and imaging data based on the ultrasound reflecting off a patient anatomy/body structure and/or an interventional instrument. The ultrasound machine 102 includes various components, some of which include the scanner 104, one or more processors 106, a display device 108, a memory 110, and a transceiver 112.

A user 114 (e.g., nurse, ultrasound technician, operator, sonographer, clinician, etc.) directs the scanner 104 toward a patient 116 to non-invasively scan internal bodily structures (e.g., patient anatomies such as organs, tissues, bones, etc.) of the patient 116 for testing, diagnostic, therapeutic, or procedural reasons, including determining venous congestion. In some embodiments, the scanner 104 includes an ultrasound transducer array and electronics communicatively coupled to the ultrasound transducer array to transmit ultrasound signals to the patient's anatomy and receive ultrasound signals reflected from the patient's anatomy. In some embodiments, the scanner 104 is an ultrasound scanner, which can also be referred to as an ultrasound probe or transducer.

In some embodiments, the scanner 104 is a multi-array scanner. For instance, a multi-array scanner in accordance with the present invention can include one or more of the arrays described in U.S. patent application Ser. No. 18/613,694 filed on Mar. 22, 2024 entitled Multi-Dimensional and Multi-Frequency Ultrasound Transducers to Zhang et al., the disclosure of which is incorporated herein by reference in its entirety. A multi-array scanner in accordance with the present invention can include one or more of the arrays described in U.S. patent application Ser. No. 17/561,313 filed on Dec. 23, 2021 entitled Array Architecture and Interconnection for Transducers to Li et al., the disclosure of which is incorporated herein by reference in its entirety. In some embodiments, where the femoral vein is added to the VExUS protocol the use of a multiarray transducer allows the entire protocol to be performed using one transducer to access the venous flow in both shallow and deep parts of the body, using the graphics on screen to guide the user through the experience.

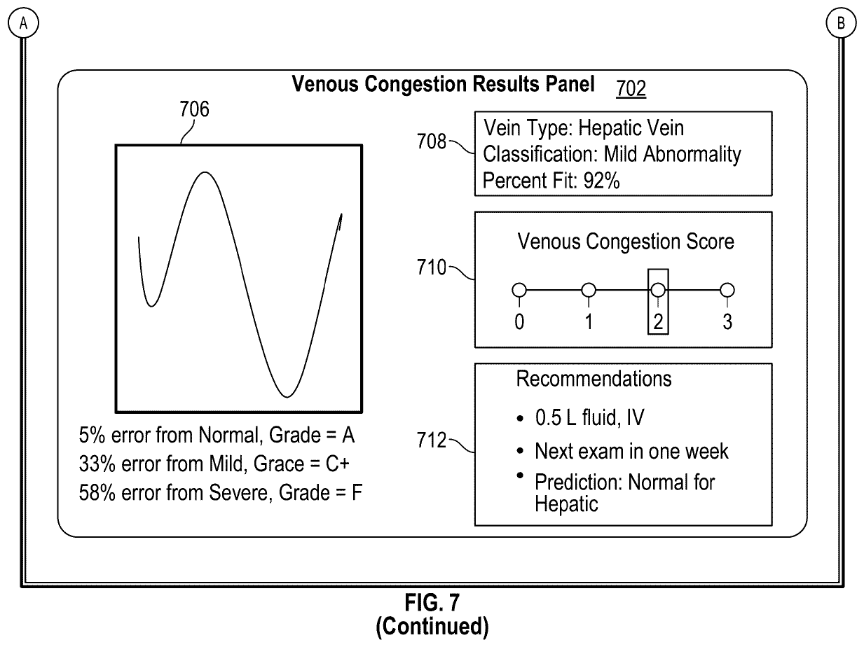

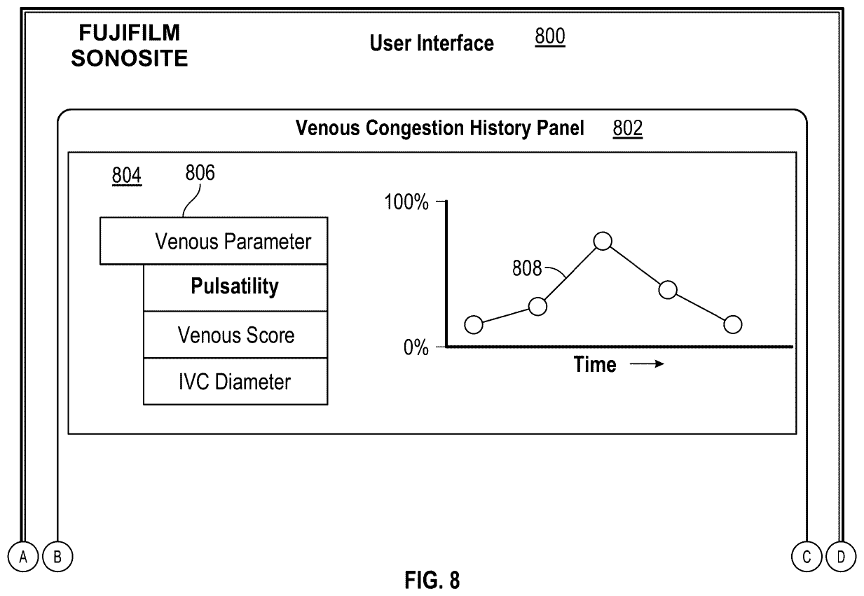

The display device 108 is coupled to the processor 106, which can include any suitable processor, number of processors, or processor system, such as one or more CPUs, GPUs, vector processors, RISC processors, CISC processors, VLIW processors, etc. The processor 106 can execute instructions stored on memory 110 to perform operations disclosed herein for determining venous congestion. For example, the processor 106 can process the reflected ultrasound signals to generate ultrasound data, including an ultrasound image. The display device 108 is configured to generate and display an ultrasound image (e.g., ultrasound image 118) of the anatomy and/or interventional instrument based on the ultrasound data generated by the processor 106 from the reflected ultrasound signals detected by the scanner 104. In some embodiments, the ultrasound data includes the ultrasound image 118 or data representing the ultrasound image 118. The transceiver 112 can be configured to transmit, e.g., over a network maintained by a care facility, the ultrasound data and/or any data related to the ultrasound examination, such as medical worksheet data, to a medical archiver (e.g., a vendor neutral archive (VNA)). In some embodiments, the transceiver 112 can receive data from the medical archiver, such as patient history data or previous examination data. In some embodiments, the previous examination data can be displayed in a user interface as part of a current examination, for example, for comparison to results obtained during the current examination, as described below in more detail with respect to FIG. 8. Additionally or alternatively, the previous examination data can be displayed in a user interface as part of patient monitoring, e.g., after an ultrasound examination.

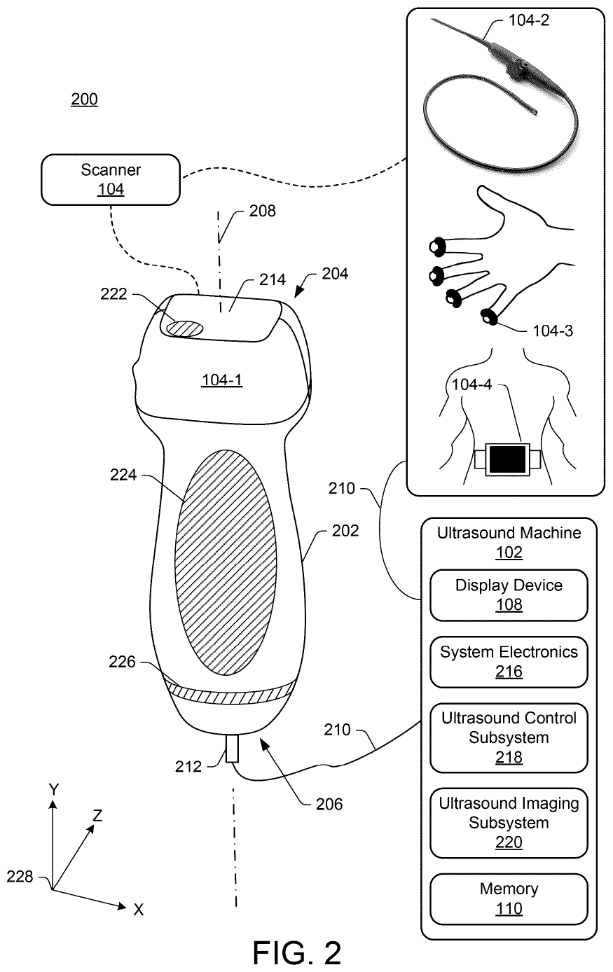

FIG. 2 illustrates an example implementation 200 of the ultrasound system illustrated in the environment 100 of FIG. 1. In the implementation 200, the scanner 104 (e.g., ultrasound scanner) can be any suitable type of ultrasound scanner. In an example, the scanner 104 includes a scanner 104-1 configured for handheld operation external to a patient's body. Other examples of the scanner 104, including scanner 104-2, 104-3, and 104-4, are discussed below. The scanner 104-1 includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104-1. The scanner 104-1 is electrically coupled to an ultrasound imaging system (e.g., the ultrasound machine 102) via a coupling 210. In some embodiments, the coupling 210 includes a cable that is attached to the proximal end portion 206 of the scanner 104-1 by a strain-relief element 212. In some embodiments, the coupling 210 includes a wireless coupling so that the scanner 104-1 is wirelessly coupled to the ultrasound imaging system and communicates with the ultrasound imaging system via one or more wireless transmitters, receivers, or transceivers over a wireless connection or network (e.g., Bluetooth™, Wi-Fi™, etc.).

A transducer assembly 214 having one or more transducer elements is electrically coupled to system electronics 216 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 216 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than 15 Megahertz (MHz)) and/or high-frequency ultrasound (e.g., greater than or equal to 15 MHz). A particular frequency range to use can readily be determined based on various factors, including, for example, depth of imaging, desired resolution, and so forth.

In some embodiments, the system electronics 216 include one or more processors (e.g., the processor(s) 106 from FIG. 1), integrated circuits, application-specific integrated circuits (ASICs), Field Programmable Gate Arrays (FPGAs), and power sources to support functioning of the ultrasound machine 102. In some embodiments, the ultrasound machine 102 also includes an ultrasound control subsystem 218 having one or more processors. At least one processor, FPGA, or ASIC can cause electrical signals to be transmitted to the transducer(s) of the scanner 104 to emit sound waves and also receives electrical pulses from the scanner 104 that were created from the returning echoes. One or more processors, FPGAs, or ASICs can process the raw data associated with the received electrical pulses and form an image that is sent to an ultrasound imaging subsystem 220, which causes the image (e.g., the image 118 in FIG. 1) to be displayed via the display device 108. Thus, the display device 108 displays ultrasound images from the ultrasound data processed by the processor(s) of the ultrasound control subsystem 218.

In some embodiments, the ultrasound machine 102 also includes one or more user input devices (e.g., a keyboard, a cursor control device, a microphone, a camera, touchscreen, etc.) that input data and enable taking measurements from the display device 108 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, and so on) for storing the acquired ultrasound data. In aspects, the disk storage device includes the memory 110, which is local to the ultrasound machine 102. Alternatively, the memory 110 used for storing the acquisition data can be remote, such as on a remote server communicatively connected to the ultrasound machine 102. In addition, the ultrasound machine 102 can include a printer that prints the image from the displayed data. To avoid obscuring the techniques described herein, such user input devices, disk storage device, and printer are not shown in FIG. 2.

In some embodiments, the ultrasound scanner 104-1 in the implementation 200 also includes one or more pressure sensors 222 on the lens of the scanner 104-1, and one or more pressure sensors 224 on the enclosure 202 of the scanner 104-1. The pressure sensors 222 and 224 can include in, on, or under a sensor region any suitable type of sensors for determining a pressure. In some embodiments, the pressure sensors 222 and 224 include capacitive sensors that can measure a capacitance, or change in capacitance, caused by a user's touch or proximity of touch, as is common in touchscreen technologies. The pressure sensors 222 and 224 can generate sensor data indicative of a touch or pressure. The sensor data can include a binary indicator that indicates the presence and absence of a touch on the sensor. For instance, a “1” for sensor data can indicate that a pressure is sensed at the pressure sensor, and a “0” for the sensor data can indicate that a pressure is not sensed at the pressure sensor. Additionally or alternatively, the sensor data can include a multi-level indicator that indicates an amount of pressure on the sensor, such as an integer scale from zero to five. For instance, a “0” can indicate that no pressure is detected at the sensor, and a “1” can indicate a small amount of pressure is detected at the sensor. A “2” can indicate a larger amount of pressure is detected at the sensor than a “1”, and a “5” can indicate a maximum amount of pressure is detected at the sensor.

The pressure sensors 222 and 224 are illustrated in FIG. 2 as ellipses for clarity, and generally can be of any suitable shape and size, and generate sensor data indicating pressure at any suitable number of points. For instance, in some embodiments, the pressure sensors 222 cover an exterior surface of the lens of the scanner 104-1 and can be used to determine when the scanner is placed against a patient. Additionally or alternatively, the pressure sensors 224 can substantially cover the enclosure 202 of the scanner 104-1 and can be used to determine when a clinician grabs the scanner 104-1 for use in an ultrasound examination (e.g., the clinician has a suitable grip on the scanner 104-1 to perform the ultrasound examination). The ultrasound system can use the sensor data from one or both of the pressure sensors 222 and 224 to generate a trigger signal that can be used for determining venous congestion with ultrasound. For instance, when the sensor data from one or both of the pressure sensors 222 and 224 is above a threshold level, and/or the sensor data from the pressure sensors 224 indicate a grip pattern indicative of a human operating the scanner, the system can generate a trigger signal. The trigger signal can be used to cause the display device 108 to display classification waveforms that can be matched to a Doppler waveform. In some embodiments, a user can select a classification waveform displayed on the display device 108. Additionally or alternatively, the system can implement a machine-learned model (e.g., a neural network) that automatically matches a Doppler waveform to a classification waveform to generate a score indicative of venous congestion. In some embodiments, the system enables the machine-learned model responsive to the trigger signal generated based on the sensor data from one or both of the pressure sensors 222 and 224. For instance, when a VExUS protocol is being performed and the pressure data indicates a threshold pressure level is met or a grip pattern indicative of a human operating the scanner, the system can enable the machine-learned model.

In some embodiments, the system enables a machine-learned model, responsive to the trigger signal generated based on the sensor data from one or both of the pressure sensors 222 and 224, to place a pulse wave Doppler gate on an ultrasound image. Additionally or alternatively, the system can enable a counter, responsive to the trigger signal generated based on the sensor data from one or both of the pressure sensors 222 and 224, to begin a countdown. Upon expiration of the countdown, the system can enable a pulse wave Doppler mode used to generate a Doppler waveform for determining venous congestion with ultrasound. The duration of the countdown can be user selectable. For instance, a user may be confident that they can acquire an ultrasound image with an acceptable view in time for the countdown to expire. The system can then switch to the pulse wave Doppler mode at a cadence suitable for (e.g., tailored to) the user, without requiring the user to manually change the mode of the system.

In some embodiments, the system can enable a machine-learned model, responsive to the trigger signal generated based on the sensor data from one or both of the pressure sensors 222 and 224, that is implemented as a blood vessel classifier. The blood vessel classifier can make a determination of a blood vessel type, such as a vein or artery, and a determination of a vein type for blood vessels classified as veins, including vein types of hepatic, portal, and renal (e.g., intrarenal).

In some embodiments, the system enables, responsive to the trigger signal generated based on the sensor data from one or both of the pressure sensors 222 and 224, a machine-learned model that generates an image quality score for an ultrasound image. The image quality score can indicate if the ultrasound image includes a suitable view of a vein for the system to generate a Doppler waveform for the vein. If the image quality score is below a threshold score, the system can prompt the user to move or readjust the scanner and acquire another imaging mode. If the image quality score is above a threshold score, the system can start the countdown that, upon expiration, causes the system to switch to a pulse wave Doppler mode.

In some embodiments, the scanner 104-1 includes an inertial measurement unit (IMU) 226 for generating positional data that determines a position and orientation of the scanner 104-1 in a coordinate system, e.g., the coordinate system 228 in FIG. 2. The IMU can include a combination of accelerometers, gyroscopes, and magnetometers, and generate positional data including data representing six degrees of freedom (6 DOF), such as yaw, pitch, and roll angles in the coordinate system. Typically, 6 DOF refers to the freedom of movement of a body in three-dimensional space. For example, the body is free to change position as forward/backward (surge), up/down (heave), left/right (sway) translation in three perpendicular axes, combined with changes in orientation through rotation about three perpendicular axes, often termed yaw (normal axis), pitch (transverse axis), and roll (longitudinal axis). Additionally or alternatively, the ultrasound system can include a camera and fiducial markers on the scanner 104-1 (not shown in FIG. 2) to determine the positional data for the ultrasound scanner 104-1. In some embodiments, the system generates, based on the positional data, a trigger signal as described above. For instance, the positional data can indicate that the scanner 104 is within a threshold distance of the patient, and the trigger signal can be used by the ultrasound system to enable one or more machine-learned models, e.g., to enable a blood vessel classifier, enable a pulse wave Doppler mode, place a pulse wave Doppler gate, generate a Doppler waveform, start a countdown, etc., as described above.

In some embodiments, the scanner 104 also includes example scanners 104-2, 104-3, and 104-4 that are coupled to the ultrasound machine via the coupling 210. The scanner 104-2 is an example of a transesophageal ultrasound (TEU) scanner. Hence, the scanner 104-2 includes an array that can be inserted into a patient's mouth and through their esophagus for imaging of veins from an internal point of view, for determining venous congestion with ultrasound. Examples of a TEU scanner are described in U.S. patent application Ser. No. 18/071,420 entitled A Transesophageal Ultrasound System to Nally et al. filed Nov. 29, 2022, the disclosure of which is incorporated herein by reference in its entirety. Additional examples of a TEU scanner are described in U.S. patent application Ser. No. 18/071,416, entitled Ultrasound Probe with Integrated Controls to Nally et al. filed Nov. 29, 2022, the disclosure of which is incorporated herein by reference in its entirety.

The scanner 104-3 is an example of a wearable ultrasound scanner that can be operator worn. The scanner 104-3 includes one or more rings that can be worn on an operator's fingers. The rings of the example scanner 104-3 can include one or more transducer arrays that can be rigid, flexible, or semi-rigid. The rings can be coupled to the ultrasound machine 102 to generate an ultrasound image based on a synthetic aperture (e.g., an aperture larger than the rings themselves, due to the space between the operator's finger tips). The operator can move their fingertips around the patient's skin to image any suitable patient anatomy, including the IVC, hepatic vein, portal vein, and intrarenal (e.g., renal) vein. In some embodiments, the scanner 104-3 can be worn by the patient for a self-administered examination.

The scanner 104-4 is an example of a wearable scanner that can be patient worn. In some embodiments, the scanner 104-4 has a form factor of one or more wearable patches that can be affixed to a patient. The patches can include one or more transducer arrays that can be rigid, flexible, or semi-rigid. The patches can remain affixed to a patient for long term monitoring (e.g., days or weeks). The system can periodically, e.g., hourly, once or twice a day, etc. generate a score indicative of venous congestion, and report the score to a medical archiver, server, nurse's station, etc. The system can implement one or more machine-learned models to automatically generate the score via ultrasound transmitted and received by one or more patches of the scanner 104-4. For instance, the system can implement a blood vessel classifier with a first machine-learned model to determine a vein type for a vein, such as a vein type identifying a hepatic vein, portal vein, or intrarenal (e.g., renal) vein. The system can then enable a pulse wave Doppler mode to generate a Doppler waveform for the vein. The system can obtain classification waveforms for the vein type, such as from a memory of the ultrasound machine 102, and can implement a second machine-learned model to determine a degree of abnormality of the Doppler waveform, such as by matching the Doppler waveform to one of the classification waveforms. The system can repeat these processes for multiple veins of different vein types. Based on the degrees of abnormality for the multiple veins, e.g., two or more of the hepatic vein, the portal vein, and the intrarenal (e.g., renal) vein, the system can generate a score indicating venous congestion (as described below in more detail).

### Example User Interfaces

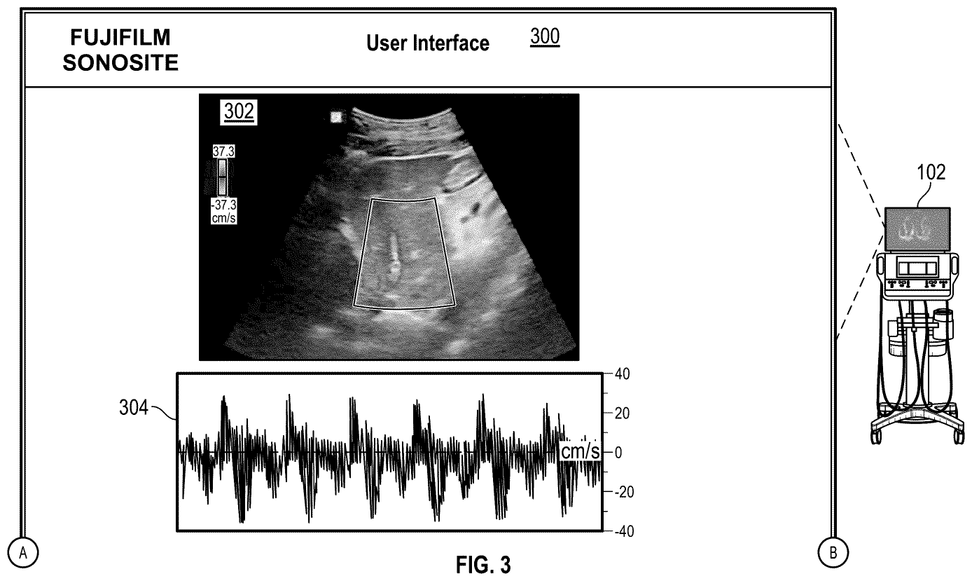

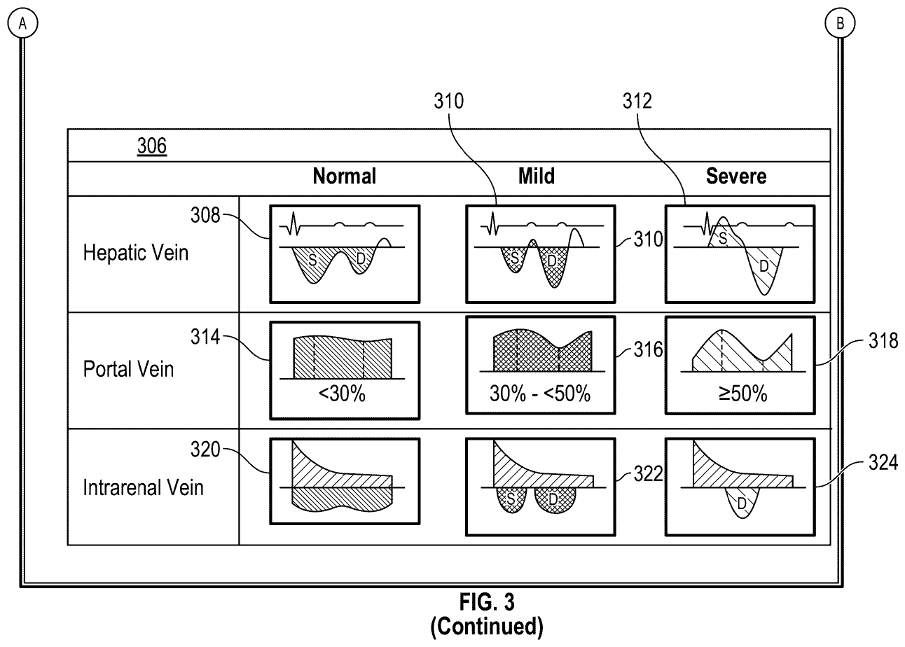

FIG. 3 illustrates an example user interface 300 of an ultrasound machine 102 for determining venous congestion with ultrasound. The user interface 300 includes an image panel 302, a Doppler waveform panel 304, and a classification waveform panel 306. The image panel 302 can display any suitable image, such as a B-mode image, a Doppler image, etc. For instance, the image displayed in the example image panel 302 of FIG. 3 includes a B-mode image with color Doppler overlays to indicate fluid flow directions.

The Doppler waveform panel 304 displays a Doppler waveform for a blood vessel (e.g., vein) included in the ultrasound image displayed in the image panel 302. The Doppler waveform of the Doppler waveform panel 304 indicates flow velocity for the blood in the vein. Positive velocity indicates flow in a direction, and negative velocity indicates flow in the opposite direction. The Doppler waveform panel 304 can display any suitable number of Doppler waveforms, such as one for a first blood vessel and another for a second blood vessel. The Doppler waveform panel 304 can overlay multiple Doppler waveforms. The system can generate a Doppler waveform by enabling a pulse wave Doppler mode and placing a pulse wave Doppler gate (not shown in FIG. 3 for clarity) on a vein in the image of the image panel 302. In some embodiments, the system automatically and without user intervention places the pulse wave Doppler gate on a vein in the image of the image panel 302. For instance, the ultrasound system can implement one or more machine-learned models trained to identify and segment blood vessels in an image, and place a pulse wave Doppler gate on a vein. In some embodiments, the system automatically labels diastole and systole periods of the Doppler waveform displayed in the Doppler waveform panel 304. For example, the system can implement one or more machine-learned models to determine and label the diastole and systole periods, such as with an “S” for systole periods and a “D” for diastole periods. In some embodiments, the system determines the diastole and systole periods based on electrocardiogram (ECG) data. In another example, the system determines the diastole and systole periods without using ECG data.

The classification waveform panel 306 displays classification waveforms 308-324 for veins including a hepatic vein, a portal vein, and an intrarenal vein. The hepatic vein can indicate venous congestion from the liver; the portal vein can indicate venous congestion from the gut; and the intrarenal vein can indicate venous congestion from the kidney. The classification waveforms indicate degrees of abnormality of a Doppler waveform. Example degrees of abnormality include “normal” (to indicate no abnormality), “mild” (to indicate mildly abnormal), and “severe” (to indicate severely abnormal). More specifically, for the hepatic vein, the classification waveform 308 represents a normal Doppler waveform, the classification waveform 310 represents a mildly abnormal Doppler waveform, and the classification waveform 312 represents a severely abnormal Doppler waveform. The classification waveforms 308-312 include an “S” label for the systole period and a “D” label for the diastole period. For the portal vein, the classification waveform 314 represents a normal Doppler waveform, the classification waveform 316 represents a mildly abnormal Doppler waveform, and the classification waveform 318 represents a severely abnormal Doppler waveform. For the intrarenal vein, the classification waveform 320 represents a normal Doppler waveform, the classification waveform 322 represents a mildly abnormal Doppler waveform, and the classification waveform 324 represents a severely abnormal Doppler waveform.

In some embodiments in FIG. 3, the Doppler waveform displayed in the Doppler waveform panel 304 is for a hepatic vein. Hence, a user can match the Doppler waveform in the Doppler waveform panel 304 to one of the classification waveforms 308-312 to determine a degree of abnormality for the Doppler waveform. The user can select one of the classification waveforms 308-312 in any suitable way, such as with a cursor on a mouse, tapping the waveform on the display when the display includes a touch screen, with a voice command, a gesture (e.g., holding up three fingers to indicate the selection of a third waveform), etc. In some embodiments, the system automatically populates a medical worksheet with the selected one of the classification waveforms responsive to the user selection. In some embodiments, the ultrasound system implements a machine-learned model to select one of the classification waveforms 308-312 that best matches the Doppler waveform in the Doppler waveform panel 304. Based on the selection of one of the classification waveforms 308-312 (e.g., a user selection or a selection from a machine-learned model), the system can determine a degree of abnormality as “normal”, “mild”, or “severe” for the Doppler waveform.

In some embodiments, these steps can be repeated for veins of other vein types. For example, an ultrasound scanner can be repositioned to image a different patient anatomy and generate an ultrasound image that includes a portal vein or an intrarenal vein. The system can be enabled in a pulse wave Doppler mode to generate Doppler waveforms for veins of the other types, and the user or system can determine degrees of abnormality for the Doppler waveforms by matching them to the classification waveforms 314-318 for a portal vein and classification waveforms 320-324 for an intrarenal vein. Based on the selected classification waveforms and/or the degrees of abnormality represented by the selected classification waveforms, the system can generate a score indicative of venous congestion, e.g., a VExUS score. The score is also based on the status of the IVC, which may be evaluated visually and/or by measuring the diameter of the IVC. Diameter measurements of the IVC may be created in 2D or M-Mode and could include either or both long and transverse IVC measurements.

In some embodiments, a VExUS score is set to zero if the IVC is non-dilated and/or the diameter (or both IVC diameters, if two are measured) is less than 2 cm. Otherwise, the VExUS score is set to one if the selected classification waveforms represent any combination of “normal” and “mild” abnormalities (e.g., the selected classification waveforms do not represent a “severe” abnormality). Otherwise, the VExUS score is set to two if the selected classification waveforms represent one and only one “severe” abnormality. Otherwise, the VExUS score is set to three if the selected classification waveforms represent two or more “severe” abnormalities. Hence, in some cases, not all of the hepatic, portal, and intrarenal veins need to be imaged. For instance, if the maximum IVC diameter is greater than 2 cm, and two of the veins result in “severe” abnormality classifications, then the VExUS score is three, and the third vein does not need to be measured/classified to determine the VExUS score. Note that the techniques disclosed herein are not limited to using these classifications and score, and can be used for any VExUS score that involves the matching of a Doppler waveform to a candidate waveform.

In the example in FIG. 3, the classification waveform panel 306 displays classification waveforms 308-324 simultaneously. In some embodiments, the ultrasound system displays only the classification waveforms for the vein type that is currently displayed in the image in the image panel 302. For instance, the system can implement a machine-learned model to determine the vein type for a vein in the image in the image panel 302, and based on the determined vein type, the system can display the classification waveforms for the vein type while suppressing display of the classification waveforms for other vein types. In some other embodiments, the system determines the vein type based on a current step of an ultrasound protocol (e.g., a VExUS protocol). For instance, if the current protocol step enables a hepatic vein examination step, then the system can display the classification waveforms for the hepatic vein while suppressing display of the classification waveforms for portal and intrarenal veins.

In some embodiments, for the portal vein, the Doppler waveform 304 can be matched to one of the classification waveforms 314-318 based on a pulsatility fraction. For instance, the classification waveform 314 corresponds to a “normal” degree of abnormality (e.g., no abnormality), and can be characterized by a pulsatility fraction of less than 30%. The classification waveform 316 corresponds to a “mild” degree of abnormality, and can be characterized by a pulsatility fraction of greater than 30% and less than 50%. The classification waveform 318 corresponds to a “severe” degree of abnormality, and can be characterized by a pulsatility fraction of greater than or equal to 50%. In some embodiments, the system generates a pulsatility fraction for the Doppler waveform, and matches one of the classification waveforms 314-318 to the Doppler waveform based on the generated pulsatility fraction. For instance, if the system generates for the Doppler waveform a pulsatility fraction of 46%, then the system selects the classification waveform 316 and a “mild” degree of abnormality for the Doppler waveform. In some embodiments, the pulsatility fraction is computed as the ratio of the difference between the minimum velocity and the maximum velocity over the maximum velocity. The minimum and maximum velocities correspond to the minimum and maximum values of the Doppler waveform, respectively. Alternatively, in some embodiments, a user selects one of the classification waveforms 314-318 for the portal vein based on visually comparing the classification waveforms 314-318 to the Doppler waveform 304, without explicitly computing the pulsatility fraction of the Doppler waveform.

In some embodiments, to improve visual comparison between one or more of the classification waveforms 308-324 to the Doppler waveform in the Doppler waveform panel 304, one or more of the classification waveforms 308-324 can be shown in the user interface panel with the Doppler waveform. In some embodiments, one or more of the classification waveforms 308-324 is shown side by side with the Doppler waveform in panel 304 for comparison purposes. In such a case, a user can select any of the classification waveforms 308-324 (e.g., touch one or more classification waveforms 308-324, pressing a graphical user interface (GUI) element in user interface 300, etc.) and, in response thereto, the system causes the classification waveform to be displayed next the Doppler waveform in the Doppler waveform panel 304. In some other embodiments, selecting one or more of the classification waveforms 308-324 causes the selected classification waveform to be superimposed on the Doppler waveform in the Doppler waveform panel 304. For example, in response to selecting the classification waveforms 308-312, the ultrasound machine (e.g., computing device) causes all three of the classification waveforms 308-312 to be superimposed onto the Doppler wave in Doppler waveform panel 304. In some embodiments, different colors or other user interface effects (e.g., highlighting, line type, etc.) are used to distinguish the individual waveforms in in Doppler waveform panel 304. Such comparisons can be beneficial to identify the classification of the Doppler waveform in Doppler waveform panel 304.

In some embodiments, to improve visual comparison between one or more of the classification waveforms 308-324 to the Doppler waveform in the Doppler waveform panel 304, the system determines where systolic (S wave) and diastolic (D wave) are located in the Doppler waveform in the Doppler waveform panel 304 and displays the S wave and D wave on the Doppler waveform in the Doppler waveform panel 304. In some embodiments, the location of S wave and D wave are automatically displayed on the Doppler waveform in the Doppler waveform panel 304. In some embodiments, the ultrasound machine uses a machine-learning architecture (such as, for example, but not limited to, one described below) or another artificial intelligence architecture to determine the location of the S wave and D wave in the Doppler waveform in the Doppler waveform panel 304 for display thereon.

In some embodiments, the ultrasound machine integrates the test results, or output, of an electrocardiogram (ECG) into the user interface 300. In some embodiments, these results are integrated into the Doppler waveform panel 304. In some other embodiments, these results are displayed in a panel in proximity to the Doppler waveform in Doppler waveform panel 304. In some embodiments, the system uses the ECG results to determine the systole and diastole periods of the Doppler waveform that the system can automatically label.

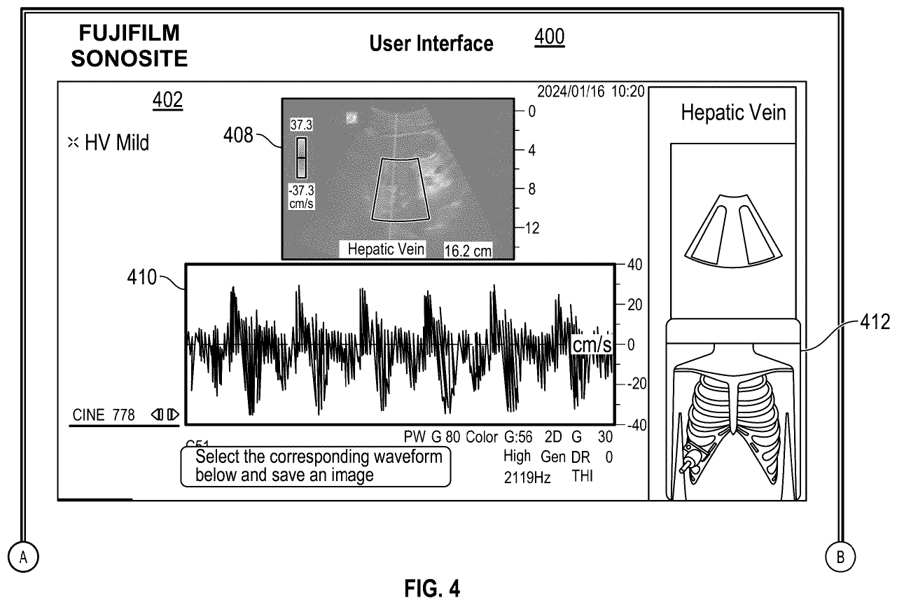

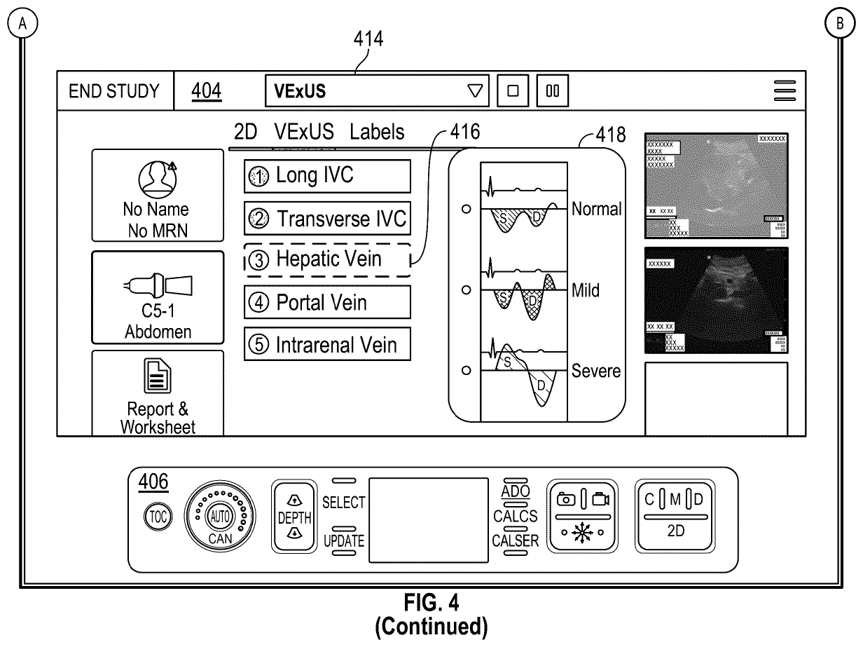

FIG. 4 illustrates some embodiments of user interface 400 of a computing device (e.g., an ultrasound machine, display device of an ultrasound machine, etc.) for determining venous congestion with ultrasound. The user interface 400 includes an ultrasound panel 402, a protocol panel 404, and a control panel 406. The ultrasound panel 402 can display any suitable image, such as an ultrasound image, a Doppler image, Doppler waveforms, a visual representation for guidance (e.g., how to hold an ultrasound scanner, an example of the expected view to obtain in an ultrasound image, etc.), and the like. In some embodiments in FIG. 4, the ultrasound panel 402 includes an ultrasound image 408, a Doppler waveform 410, and a visual representation 412 of probe guidance.

The ultrasound image 408 includes a vein with color Doppler overlays to indicate blood flow, and a pulse wave Doppler gate has been placed on the image. Based on the pulse wave Doppler gate, with the system enabled in a pulse wave Doppler mode, the system generates the Doppler waveform 410 that indicates flow velocity in the vein (e.g., at the location of the pulse wave Doppler gate) over time. To instruct the user to obtain the proper view in the ultrasound image 408, the visual representation 412 indicates how to orient the scanner (e.g., the visual representation 412 includes an icon of a scanner with a registration mark) and where to place the scanner on the patient.

The protocol panel 404 can include any suitable controls or options for configuring the system to operate according to an ultrasound protocol. Examples of ultrasound protocols include Focused Assessment with Sonography in Trauma (FAST), Rapid Ultrasound for Shock and Hypotension (RUSH), and VExUS. The protocol panel 404 can also include any suitable data related to an ultrasound protocol, such as patient data, a medical worksheet, images (e.g., thumbnail images of ultrasound images acquired as part of the ultrasound protocol or from a patient history or medical record), indications of ultrasound equipment (e.g., recommended scanners, scanners in use, etc.), and the like.

In some embodiments in FIG. 4, the protocol panel 404 indicates at 414 that a VExUS protocol has been selected. Accordingly, the protocol panel 404 displays options to select protocol steps “Long IVC”, “Transverse IVC”, “Hepatic Vein”, “Portal Vein”, and “Intrarenal Vein”, and the hepatic vein is selected at 416. The protocol panel 404 also includes at panel 418 classification waveforms of “normal”, “mild”, and “severe” degrees of abnormality for the hepatic vein. In some embodiments, the panel 418 displays classification waveforms for the hepatic vein, since it is selected at 416, and does not display classification waveforms for the unselected portal and intrarenal veins. By displaying only the classification waveforms for the selected vein of the protocol (e.g., the hepatic vein), the user interface is not cluttered, and the user can match the Doppler waveform 410 to one of the classification waveforms in the panel 418 without being confused or distracted by the display of waveforms that are not needed at the current step of the protocol.

In some embodiments, the ultrasound system implements a machine-learned model to determine a vein type for the vein in the ultrasound image 408, and displays the classification waveforms in the panel 418 responsive to determining the vein type (e.g., hepatic). In some other embodiments, the ultrasound system determines the vein type from the selected protocol step, e.g., “hepatic vein” at 416, and displays the classification waveforms in the panel 418 responsive to determining the vein type (e.g., hepatic).

The control panel 406 can include any suitable controls and settings for controlling an ultrasound system, such as depth and gain adjustments, and a button to store images and/or video clips. The ultrasound control panel 406 can also include icons to select examination presets, such as a heart icon for a cardiac preset, a lung icon for a respiratory preset, an eye icon for an ocular present, and a leg icon for a muscular-skeletal preset (not shown for clarity). The ultrasound control panel 406 can also include options to enable one or more machine-learned models (e.g., neural networks) for processing of an ultrasound image, such as an ultrasound image displayed in the ultrasound panel 402. For instance, a cardiac neural network can be enabled to generate a value of ejection fraction, a free fluid network can be enabled to generate a segmentation of free fluid in an ultrasound image, and a pneumothorax (PTX) neural network can be enabled to generate a probability of a pneumothorax condition or collapsed lung. Other examples of machine-learned models include neural networks trained as blood vessel classifiers, a machine-learned model trained to place a pulse wave Doppler gate on a blood vessel identified by a blood vessel classifier, a machine-learned model trained to select a classification waveform based on a Doppler waveform, etc.

In some embodiments, the control panel 406 includes options to enable one or more machine-learned models for use during an ultrasound examination to determine a score indicative of venous congestion, such as a VExUS protocol that generates a VExUS score. A first machine-learned model can be implemented to determine a blood vessel type, such as a vein or artery. Another (or the same) machine-learned model can be implemented to determine a vein type for blood vessels classified as veins, include vein types of hepatic, portal, and intrarenal (e.g., renal). A machine-learned model can also be implemented to generate a waveform trace from a Doppler waveform. For example, the Doppler waveform 410 can be noisy and difficult to see for a user, and hence the user may struggle to match the Doppler waveform 410 to one of the classification waveforms in the panel 418. Hence, the machine-learned model can generate, based on the Doppler waveform 410, a waveform trace that best fits the Doppler waveform 410 (e.g., a waveform whose difference in a least-squares sense with the Doppler waveform 410 is minimized) and/or removes noise from the Doppler waveform. The user interface 400 can then display the machine-generated waveform trace (not shown in FIG. 4 for clarity), so that the user can more easily match it to one of the classification waveforms in the panel 418.

Some embodiments of a machine-learned model that can be implemented by the ultrasound system and enabled via the control panel 406 include a machine-learned model that processes Doppler waveforms for different vein types (e.g., hepatic, portal, and renal), and automatically and without user intervention generates a score indicative of venous congestion, e.g., a VExUS score. The machine-learned model can include layers that first generate waveform traces of the Doppler waveforms, as described in the previous paragraph, and subsequent layers to process the waveform traces and generate the score. In some embodiments, the machine-learned model generates a degree of fit (e.g., a measure of the match) of the Doppler waveforms and/or the waveform traces to one or more of the classification waveforms. For instance, the degree of fit can include a percent error between two waveforms, a grade (e.g., a letter grade or number on a scale, such as 1-10, etc.).

Some other embodiments of a machine-learned model that can be implemented by the ultrasound system and enabled via the control panel 406 include a machine-learned model that automatically sets a scale (e.g., a time scale) for display of the Doppler waveform 410. For example, the machine-learned model can process Doppler data representing the Doppler waveform 410 so that its display includes a minimum and/or maximum number of cycles of the waveform. Additionally or alternatively, the ultrasound system can determine a scale (e.g., a time scale) for the display of the Doppler waveform 410 based on the vein type determined, as described above.

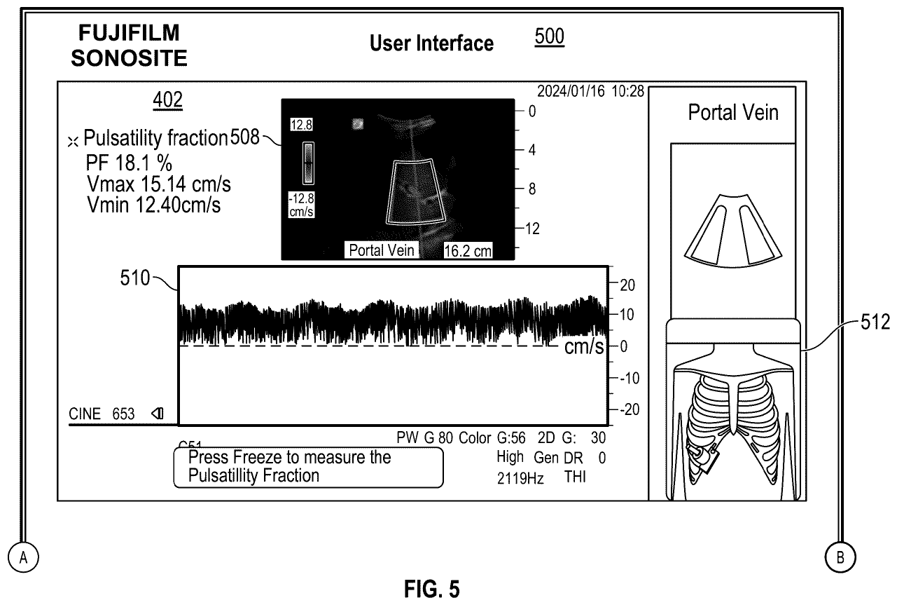

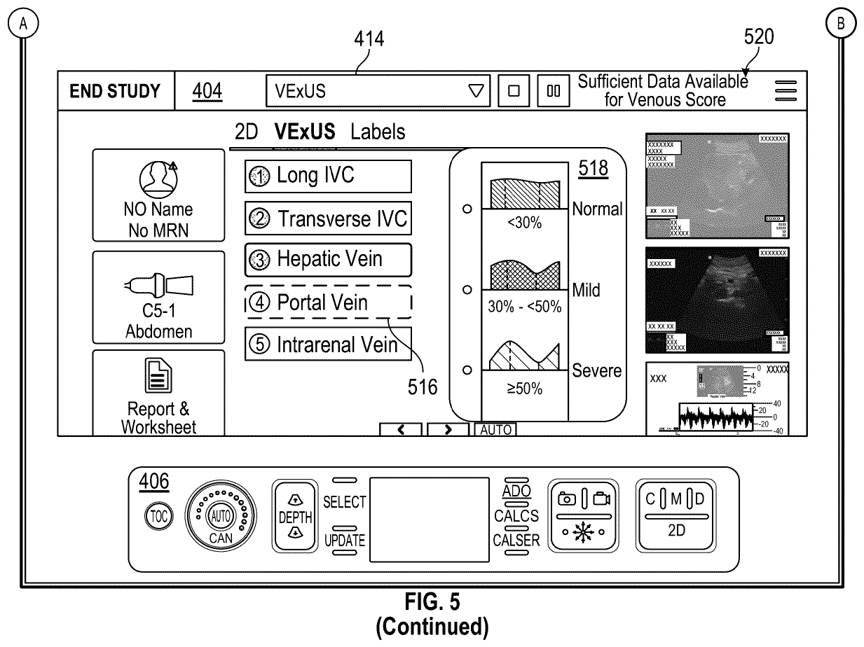

FIG. 5 illustrates some embodiments of user interface 500 of a computing device (e.g., an ultrasound machine, display device of an ultrasound machine, etc.) for determining venous congestion with ultrasound. The user interface 500 includes the ultrasound panel 402, the protocol panel 404, and the control panel 406, as described above with respect to FIG. 4. However, in the embodiments of FIG. 5, a portal vein is selected as part of a VExUS protocol, rather than the hepatic vein as illustrated in FIG. 4. Referring to FIG. 5, the ultrasound panel 402 displays an ultrasound image 508 that includes a portal vein, and the Doppler waveform 510 for the portal vein that the system has generated in a pulse wave Doppler mode. Further, the ultrasound panel 402 includes a visual representation 512 that illustrates a scanner orientation and placement to acquire the ultrasound image 508 that includes the portal vein.

The protocol panel 404 indicates at 516 that the “portal vein” step of the VExUS protocol is selected. Accordingly, the system displays in the panel 518 classification waveforms for the portal vein. In some embodiments, the panel 518 in the user interface 500 of FIG. 5 and the panel 418 in the user interface 400 of FIG. 4 are at the same location of the user interfaces, and the ultrasound system removes the display of the classification waveforms for the hepatic vein and replaces them with the display of the classification waveforms for the portal vein responsive to the protocol step changing from selection of the hepatic vein at 416 to selection of the portal vein at 516. As mentioned above, in this embodiment, the user is presented only the classification waveforms for the vein type corresponding to the current protocol step and/or the vein type of the vein displayed in the user interface, thereby removing clutter from the user interface and increasing ease of use (e.g., by reducing confusion and removing unnecessary options for the current protocol step).

In some embodiments, the protocol panel 404 in the user interface 500 also indicates at 520 that sufficient data is available to generate a venous score (e.g., a VExUS score). The protocol panel 404 can indicate this condition in any suitable way, such as with text as is done at 520, with a gauge showing a fill level, a meter, a number, displaying the venous score, etc. The condition that sufficient data is available to generate a venous score can be based on an IVC diameter measurement, a number of veins and vein types that have been classified according to degrees of abnormality, the degrees of abnormality, and combinations thereof, as described above with respect to FIG. 3.

In some embodiments, the protocol panel 404 also displays a pulsatility fraction for the Doppler waveform 510. For instance, in some embodiments, the system can generate a pulsatility fraction for the Doppler waveform 510, and display its value proximate to one of the classification waveforms in the panel 518 to indicate that the one of the classification waveforms best matches the Doppler waveform 510. A user can then select the one of the classification waveforms for the portal vein step of the protocol, or the system can automatically select the one of the classification waveforms for the portal vein step of the protocol.

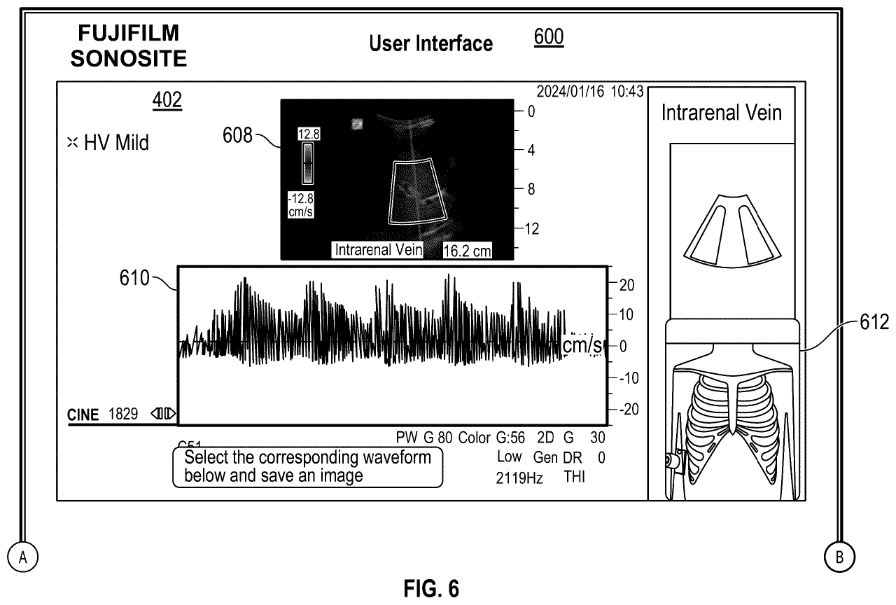

FIG. 6 illustrates some embodiments of user interface 600 of a computing device (e.g., an ultrasound machine, display device of an ultrasound machine, etc.) for determining venous congestion with ultrasound. The user interface 600 includes the ultrasound panel 402, the protocol panel 404, and the control panel 406, as described above with respect to FIGS. 4 and 5. However, in the embodiments of FIG. 6, an intrarenal vein is selected as part of a VExUS protocol, rather than the hepatic vein as illustrated in FIG. 4 and the portal vein as illustrated in FIG. 5. Hence, the ultrasound panel 402 displays an ultrasound image 608 that includes an intrarenal vein, and the Doppler waveform 610 for the intrarenal vein that the system has generated in a pulse wave Doppler mode. Further, the ultrasound panel 402 includes a visual representation 612 that illustrates a scanner orientation and placement to acquire the ultrasound image 608 that includes the intrarenal vein.

In some embodiments, the protocol panel 404 indicates at 616 that the “intrarenal vein” step of the VExUS protocol is selected. Accordingly, the protocol panel 404 displays in the panel 618 classification waveforms 620 for the intrarenal vein. Unlike the panel 418 in FIG. 4 and the panel 518 in FIG. 5, the panel 618 in FIG. 6 includes also classification waveforms for vein types not selected as part of the current protocol step, specifically classification waveforms 622 for the hepatic vein and classification waveforms 624 for the portal vein. To denote that the classification waveforms 620 for the intrarenal vein correspond to the current protocol step at the selection 616, the panel 618 emphasizes the classification waveforms 620 over the classification waveforms 622 and 624 by displaying the classification waveforms 620 larger than the classification waveforms 622 and 624. The system can implement this emphasis in any suitable way, such as, for example, but not limited to, by greying out or making opaque the classification waveforms 622 and 624, making the classification waveforms 620 brighter than the classification waveforms 622 and 624, drawing a border around the classification waveforms 620, causing the classification waveforms 620 to blink, and the like. This emphasis of the classification waveforms 620 helps to reduce clutter from the user interface and increase ease of use (e.g., helping to focus the user's attention to the area of the protocol panel 404 needed to complete the current protocol step).

In the embodiments in FIG. 6, a user has selected one of the classification waveforms 620 for the intrarenal vein displayed in the panel 618, as evidenced by the fingerprint 626. For example, a user can touch a touchscreen that implements the user interface 600, speak a voice command, perform a gesture (e.g., hold up one finger to denote a selection of the first displayed classification waveform of the classification waveforms 620), etc., to make the selection indicated by the fingerprint 626. Responsive to the selection, the system can generate a score indicative of venous congestion, e.g., a VExUS grade (as is displayed in the panel 618). If the system does not yet have enough data to generate a score, the system can enter the selection into a data queue that stores data components necessary for generation of the score, and compute the score when sufficient data is collected (e.g., when enough veins are classified).

Additionally or alternatively, the system can, responsive to the selection indicated by the fingerprint 626, populate a medical worksheet with data representing the selection, such as a score, the selected classification waveform, a degree of abnormality represented by the selected classification waveform, automatically label the ultrasound image 608 and/or the Doppler waveform 610 (e.g., to denote the selection, such as by placing text “Normal” on the ultrasound image 608 and/or the Doppler waveform 610), etc. In some embodiments, responsive to the selection indicated by the fingerprint 626, the system automatically advances the step of the protocol to a next step, such as by moving from the intrarenal vein step to a portal vein step, or any suitable step that has not yet been performed for the protocol. In some embodiments, the system determines the next step based on an operator's preference. For instance, the system can store user preferences that include an order of protocol steps, and select the next step in accordance with the order stored by the system for the current user of the system.

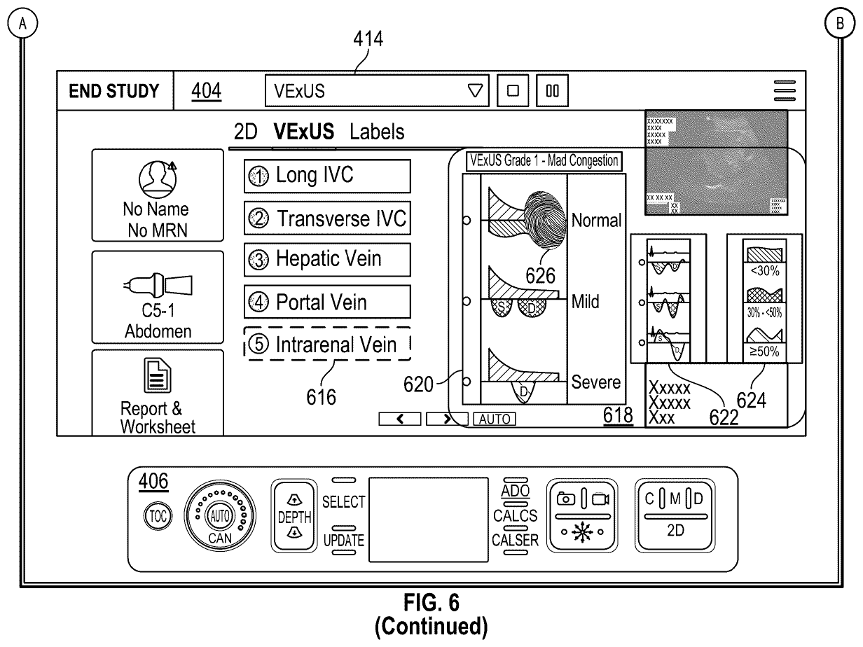

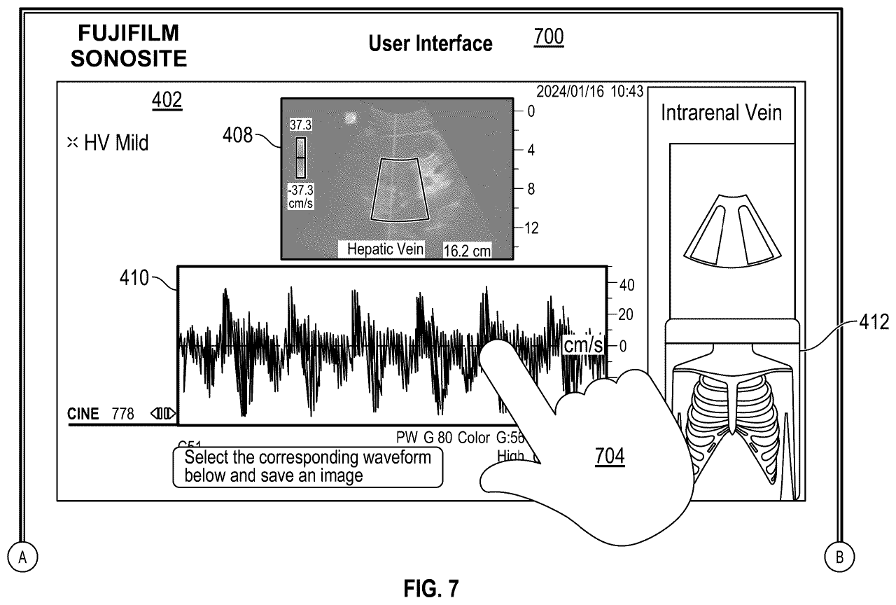

FIG. 7 illustrates some embodiments of user interface 700 of a computing device (e.g., an ultrasound machine, display device of an ultrasound machine, etc.) for determining venous congestion with ultrasound. The user interface 700 includes the ultrasound panel 402 as described above with respect to FIG. 4. As such, the ultrasound panel 402 includes the ultrasound image 408, the Doppler waveform 410, and the visual representation 412 of probe guidance. The user interface 700 also includes a venous congestion results panel 702.

As described previously, the Doppler waveform 410 can be noisy, and/or include multiple Doppler waveforms simultaneously, making it difficult for the user to visually match the Doppler waveform 410 to one of the classification waveforms to generate a venous score, e.g., a score indicative of venous congestion, such as a VExUS score. Hence, in some embodiments, the system can implement a machine-learned model to generate a waveform trace of the Doppler waveform. The machine-learned model can remove noise from the Doppler waveform 410 to generate the waveform trace that can be more easily matched by the user to a classification waveform.

Further, in some embodiments, the ultrasound system can be implemented to receive a user trace of the Doppler waveform 410, as illustrated by the hand 704 in FIG. 7. The user trace can be generated by the user touching a touch screen and tracing with their finger or a stylus the Doppler waveform 410. In some embodiments, the user can generate the user trace with a cursor controlled by a mouse coupled to the ultrasound machine.

In some embodiments, the venous congestion results panel 702 displays any suitable data related to determining venous congestion with ultrasound. For example, the venous congestion results panel 702 displays the user trace 706 of the Doppler waveform 410 generated by the hand 704. The system can determine, e.g., with a machine-learned model, one of the classification waveforms (e.g., one of 308-324) that best matches the user trace 706, e.g., in a least-squares sense. Additionally or alternatively, the user can select one of the classification waveforms based on visually comparing the user trace 706 to the classification waveforms. To assist the user, the system can generate a measure of the match of the user trace 706 to classification waveforms. For example, the venous congestion results panel 702 displays under the user trace 706 that the user trace 706 has a 5% error from a classification waveform representing a “normal” degree of abnormality, and assigns the match an A grade. The venous congestion results panel 702 also indicates that the user trace 706 has a 33% error from a classification waveform representing a “mild” degree of abnormality, and assigns the match a C+ grade. The venous congestion results panel 702 further indicates that the user trace 706 has a 58% error from a classification waveform representing a “severe” degree of abnormality, and assigns the match an F grade.

In some embodiments, the venous congestion results panel 702 also includes classification results 708, which can include any suitable data regarding classifying Doppler waveforms and determining venous congestion based on the classifying, as well as classifying 2D and M-Mode images for IVC. The classification results 708 in FIG. 7 include a vein type that has been classified, such as the hepatic vein. The classification results 708 indicate that the hepatic vein has been classified with a mild abnormality in its Doppler waveform, and that the Doppler waveform for the hepatic vein and used for the classification has a 92% fit with the classification waveform matched to the Doppler waveform. The classification results 708 can include results for any number of veins, and are limited in the example in FIG. 7 to a single hepatic vein for clarity.

In some embodiments, the venous congestion results panel 702 also includes a venous congestion score panel 710. In this example, the venous congestion score panel 710 includes a range of possible scores, including the integers 0-3. The current venous congestion score is indicated by the rectangle surrounding the numeral 2. In embodiments, the venous congestion score panel 710 displays the score as it is generated by the system, e.g., responsive to sufficient data being saved to generate the score. When the system updates the score, such as when more data becomes available (e.g., another vein is classified), then the venous congestion score panel 710 can update the score with its current value, e.g., by moving the rectangle from the numeral 2 to another number on the range of the venous congestion score panel 710.

In some embodiments, the venous congestion results panel 702 also includes a recommendation panel 712. The recommendation panel 712 can include recommendations based on determining venous congestion with ultrasound. Examples of recommendations made by the system that can be displayed in the recommendation panel 712 include to add fluid to a patient, to remove fluid, an amount of fluid to add or remove, not to change the fluid status of the patient, to monitor the patient, a date or date range for a next examination, a predicted result for a next examination, a dietary change, and the like. In the example in FIG. 7, the recommendation panel 712 indicates recommendations to add 0.5 L of fluid intravenously, to schedule an examination for one week from the present time, and that at the next examination, the hepatic vein is predicted to be classified as “normal” (e.g., no abnormality for the Doppler waveform of the hepatic vein). In some embodiments, the recommendation panel 712 be based on current and/or previous exams, which can facilitate monitoring and/or provide an indication of some type of trending.

FIG. 8 illustrates some embodiments of user interface 800 of a computing device (e.g., an ultrasound machine, display device of an ultrasound machine, etc.) for determining venous congestion with ultrasound. Referring to FIG. 8, the user interface 800 includes a venous congestion history panel 802 that can display any suitable data from a current ultrasound examination and/or one or more previous ultrasound examinations. The venous congestion history panel 802 includes a parameter panel 804 that can display any suitable parameter regarding determining venous congestion with ultrasound, in any suitable way. For example, the parameter panel 804 includes a pull-down menu 806 to select one or more parameters. In the example in FIG. 8, the pull-down menu 806 includes venous parameters for pulsatility, venous score, and IVC diameter, with pulsatility being selected (as evidenced by the bold text). Responsive to the selection of pulsatility in the pull-down menu 806, the parameter panel 804 displays the graph 808 that includes values for the pulsatility fraction over the most recent five ultrasound examinations.

In some embodiments, the venous congestion history panel 802 also includes a visual representation 810 of a history of ultrasound examinations, e.g., ultrasound examinations for determining venous congestion with ultrasound. In the example in FIG. 8, the visual representation 810 includes a time line with dates of the five most recent ultrasound examinations. A user can select one or more of the examinations indicated by the visual representation 810, and the venous congestion history panel 802 can display data from the selected examinations. In the example in FIG. 8, a user has selected the ultrasound examination dated Jun. 2, 2024, as evidenced by the fingerprint 812. For instance, the user can make the selection with a touch input. Responsive to the user selection of the examination indicated by the fingerprint 812, the venous congestion history panel 802 displays examination data 814 that was generated during the selected examination of Jun. 2, 2024. In some embodiments, the examination data 814 can be displayed simultaneously with examination data from a current examination, to enable a visual comparison. The data 814 can include any suitable data from an ultrasound examination, and can be configured by a user. For example, a user can go through a checklist of available data (not shown in FIG. 8 for clarity) and enable what data on the checklist they want to retrieve and display. In the example in FIG. 8, the data 814 includes Doppler waveforms for hepatic, portal, and renal veins. The user can move the Doppler waveforms anywhere on the user interface 800, e.g., by dragging and dropping them. Hence, the user can place the Doppler waveforms near (underneath, next to, etc.) a Doppler waveform for a current examination, for comparison. In some other embodiment, the user can arrange the Doppler waveforms sequentially, to see the progression of the waveforms over time.

Note that while the user interfaces disclosed herein display and/or present options for veins such as “Hepatic Vein”, “Portal Vein”, and “Intrarenal Vein”, the techniques disclosed herein are not limited to those vein types. For example, in some embodiments, the ultrasound machine displays other types of veins (e.g., femoral vein, etc.) in the user interface (e.g., user interfaces 300, 400, 500, 600, 700 and 800). Furthermore, the techniques disclosed herein are not limited to one type of view of the IVC. That is, in some embodiments, the user interface disclosed herein (e.g., user interfaces 300, 400, 500, 600, 700 and 800) can display many possible views of the IVC.

### Example Systems

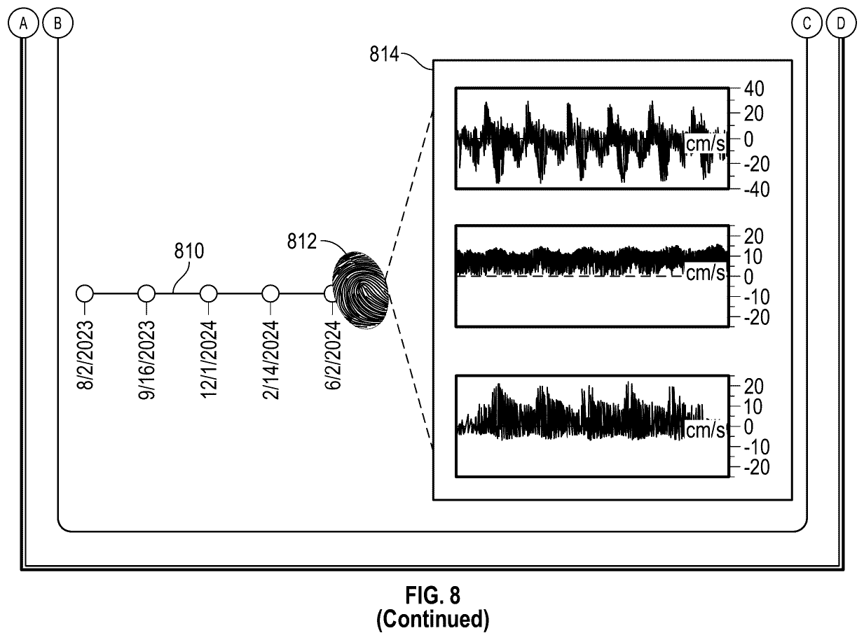

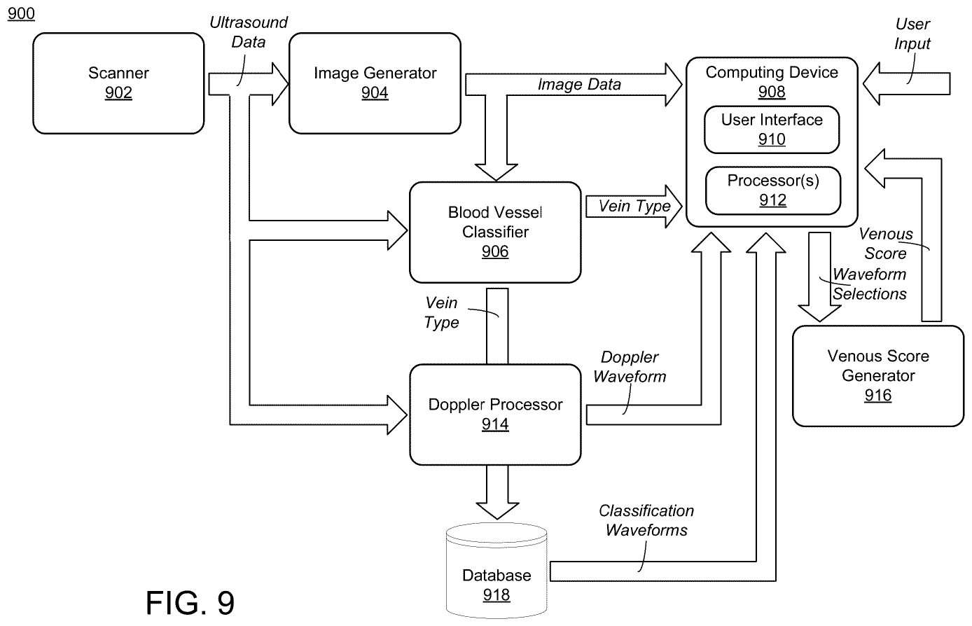

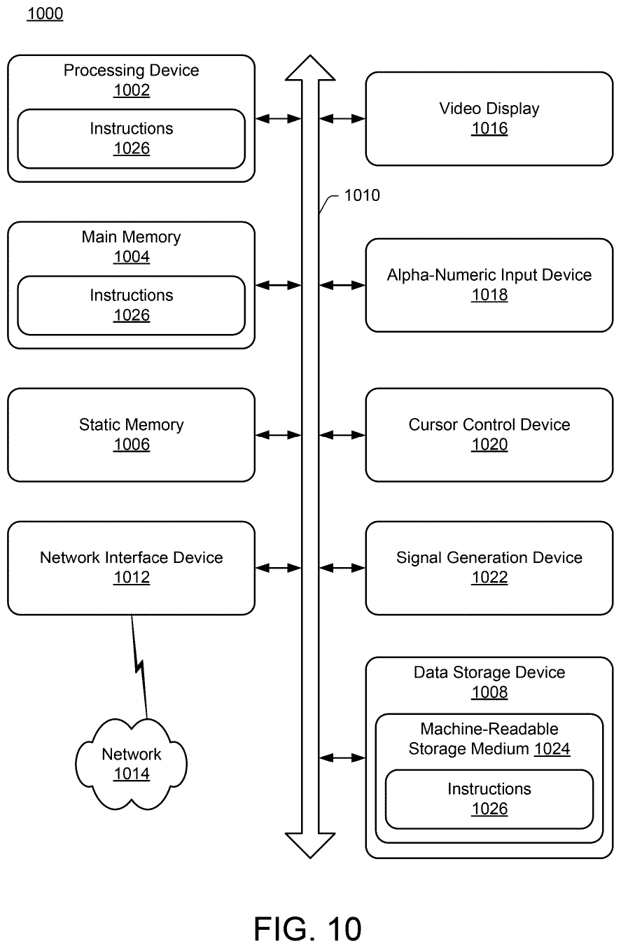

FIG. 9 illustrates an example ultrasound system 900 for determining venous congestion with ultrasound in accordance with the some embodiments. The ultrasound system 900 is an example of the ultrasound system illustrated in the environment 100 of FIG. 1 and the implementation 200 in FIG. 2. The ultrasound system 900 includes an ultrasound scanner 902, which is an example of the ultrasound scanner 104 as previously described. The ultrasound system 900 also includes an image generator 904, a blood vessel classifier 906, and a computing device 908. The computing device 908 is an example of the computing device 1000 described below with respect to FIG. 10, and of the ultrasound machine 102. The computing device 908 includes a user interface 910, which is an example of the user interfaces 300-800, and one or more processors 912, which are an example of the processors 106. The ultrasound system 900 also includes a Doppler processor 914, a venous score generator 916, and a database 918.

The ultrasound scanner 902 transmits ultrasound at a patient anatomy and generates, based on the ultrasound reflections from the patient anatomy, ultrasound data. The scanner 902 provides the ultrasound data to the image generator 904, the blood vessel classifier 906, and the Doppler processor 914. The scanner 902 can be configured in any suitable imaging mode, such as a B-mode imaging mode, a color Doppler mode, a pulse wave Doppler mode, etc. Hence, the ultrasound data generated by the scanner 902 can correspond to one or more imaging modes.

The image generator 904 receives the ultrasound data from the scanner 902. In an example, the image generator 904 receives ultrasound data when the system is in a B-mode imaging mode. The image generator 904 generates image data from the ultrasound data, such as image data representing a B-mode ultrasound image. The image generator 904 provides the image data to the computing device 908 and the blood vessel classifier 906. The computing device 908 can generate a B-mode image with the processors 912 based on the image data, and display the B-mode ultrasound image on the user interface 910.

The blood vessel classifier 906 receives the image data from the image generator 904 and, in some embodiments, the ultrasound data from the ultrasound scanner 902. The blood vessel classifier 906 can determine if the image data includes a blood vessel, determine if the blood vessel is a vein or an artery, and determine a vein type for a blood vessel that is determined to be a vein, the vein type selected from the group consisting of a hepatic vein, a portal vein, an intrarenal vein, renal vein, other types of veins or arteries. In some embodiments, the blood vessel classifier 906 implements one or more machine-learned models to make the determinations. In some embodiments, the blood vessel classifier 906 processes the ultrasound data from the scanner 902, instead of or in combination with the image data from the image generator 904. Hence, the blood vessel classifier 906 can be configured to process non-scan-converted data as well as rasterized image data. The blood vessel classifier 906 provides the vein type to the computing device 908 and the database 918.

In some embodiment the computing device 908 receives the vein type from the blood vessel classifier 906, and with the processors 912, determines classification waveforms corresponding to the vein type. The computing device 908 retrieves the classification waveforms from the database 918. Additionally or alternatively, the computing device 908 can retrieve the classification waveforms from a memory of the computing device 908 (not shown for clarity). In some embodiment the computing device 908 can configure the user interface 910 based on the vein type, including to display the classification waveforms for the vein type and remove from display other classification waveforms that do not correspond to the vein type.

The Doppler processor 914 receives the ultrasound data from the ultrasound scanner 902. In an example, the Doppler processor 914 receives ultrasound data when the system is in a pulse wave Doppler imaging mode. Based on the ultrasound data, the Doppler processor 914 generates a Doppler waveform, such as the Doppler waveforms 304, 410, 510 and 610. The Doppler processor 914 provides the Doppler waveform to the computing device 908, which can display the Doppler waveform on the user interface 910.

The computing device 908 receives a user input indicating a selection of one of the classification waveforms displayed on the user interface 910. For instance, a user can match the Doppler waveform generated by the Doppler processor 914 and displayed on the user interface 910 to one of the displayed classification waveforms. The computing device 908 provides waveform selections to the venous score generator 916. The waveform selections can include an index or label for the selected classification waveform, as well as a degree of abnormality for the selected classification waveform, and an indicator of the vein type. Based on the waveform selections, the venous score generator 916 generates and/or updates a score indicative of venous congestion, e.g., a VExUS score or venous score, and provides the score to the computing device 908. The computing device can display the score on the user interface 910, including to update a score that is already displayed with an updated score generated by the venous score generator 916.

In some embodiments, the user input provided to the computing device 908 is not used, and instead is replaced with an input from a machine-learned model implemented by the processors 912. For example, the processors 912 can implement a machine-learned model to make a selection of a classification waveform, together with its degree of abnormality, that best matches a Doppler waveform generated by the Doppler processor 914. Further, as described above, the ultrasound system can implement any suitable machine-learned model, including models to automatically place a pulse wave Doppler gate, models to classify blood vessels, models to match waveforms, and the like. Accordingly, in some embodiments, a user merely needs to place the scanner 902 to image the appropriate regions for a hepatic vein, portal vein, renal vein, and IVC, and the system can automatically generate a score indicative of venous congestion, including the details of the intermediate results (e.g., degrees of abnormality) that make up the generation of the score.

### Example Devices

FIG. 10 illustrates a block diagram of some embodiments of computing device 1000 that can perform one or more of the operations described herein. The computing device 1000 can be connected to other computing devices in a local area network (LAN), an intranet, an extranet, and/or the Internet. The computing device can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, an ultrasound machine, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some embodiments, the computing device 1000 is one or more of an ultrasound machine, an ultrasound scanner, an access point, a charging station, and a medical archiver.

The example computing device 1000 can include a processing device 1002 (e.g., a general-purpose processor, a programmable logic device (PLD), etc.), a main memory 1004 (e.g., synchronous dynamic random-access memory (DRAM), read-only memory (ROM), etc.), and a static memory 1006 (e.g., flash memory, a data storage device 1008, etc.), which can communicate with each other via a bus 1010. The processing device 1002 can be provided by one or more general-purpose processing devices such as a microprocessor, a central processing unit, or the like. In an illustrative example, the processing device 1002 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1002 can also comprise one or more special-purpose processing devices such as an application-specific integrated circuit (ASIC), a field programmable gate array (FPGA), a digital signal processor (DSP), a network processor, or the like. The processing device 1002 can be configured to execute the operations described herein, in accordance with one or more aspects of the present disclosure, for performing the operations and steps discussed herein.

The computing device 1000 can further include a network interface device 1012, which can communicate with a network 1014. The computing device 1000 also can include a video display unit 1016 (e.g., a liquid crystal display (LCD), an organic light-emitting diode (OLED), a cathode ray tube (CRT), etc.), an alphanumeric input device 1018 (e.g., a keyboard), a cursor control device 1020 (e.g., a mouse), and an acoustic signal generation device 1022 (e.g., a speaker, a microphone, etc.). In one embodiment, the video display unit 1016, the alphanumeric input device 1018, and the cursor control device 1020 can be combined into a single component or device (e.g., an LCD touch screen).

The data storage device 1008 can include a computer-readable storage medium 1024 on which can be stored one or more sets of instructions 1026 (e.g., instructions for carrying out the operations described herein, in accordance with one or more aspects of the present disclosure). The instructions 1026 can also reside, completely or at least partially, within the main memory 1004 and/or within the processing device 1002 during execution thereof by the computing device 1000, where the main memory 1004 and the processing device 1002 also constitute computer-readable media. The instructions can further be transmitted or received over the network 1014 via the network interface device 1012.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein are embodied in the data storage device 1008 of the computing device 1000 as executable instructions or code. Although represented as software implementations, the described modules can be implemented as any form of a control application, software application, signal processing and control module, hardware, or firmware installed on the computing device 1000.

While the computer-readable storage medium 1024 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### Example Environments

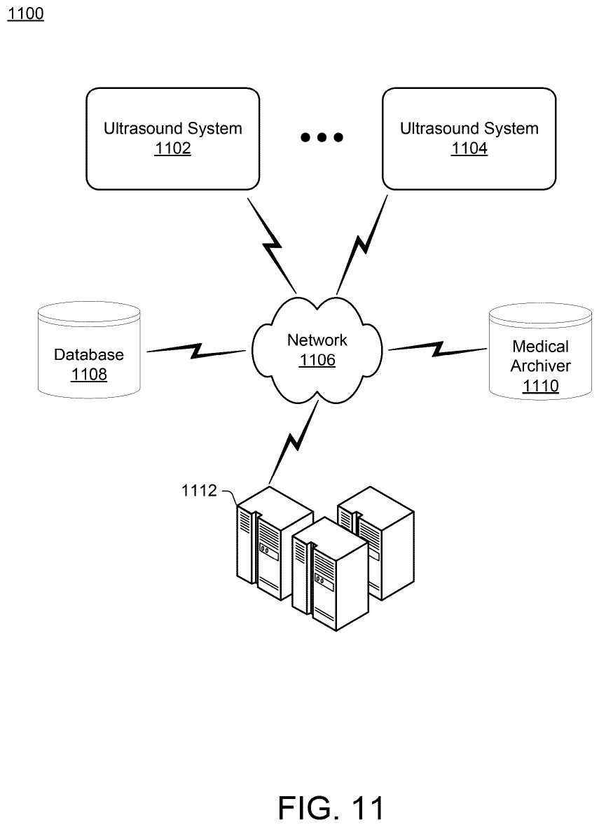

FIG. 11 illustrates an environment 1100 for an ultrasound system in accordance with some embodiments. The environment 1100 includes an ultrasound system 1102 and an ultrasound system 1104. Two example ultrasound systems 1102 and 1104 are illustrated in FIG. 11 for clarity. However, the environment 1100 can include any suitable number of ultrasound systems, such as the ultrasound systems maintained by a care facility or the department of a care facility. Generally, an ultrasound system can include any suitable device (e.g., a component of an ultrasound system). Examples devices of the ultrasound systems 1102 and 1104 include a charging station, an ultrasound machine, a display device (e.g., a tablet or smartphone), an ultrasound scanner, and an ultrasound cart. Other examples include a transducer cable, a transducer cable holder, a docking station for an ultrasound machine, a scanner station configured to hold one or more ultrasound scanners, a needle guide, a battery for a wireless ultrasound scanner, a battery for an ultrasound machine, a registration system, and the like.

The ultrasound systems 1102 and 1104 can be in communication via the network 1106 as part of the environment 1100. The network 1106 can include any suitable network, such as a local area network, a wide area network, a near field communication network, the Internet, an intranet, an extranet, a system bus that couples devices or device components (e.g., in an ASIC, FPGA, or SOC), and combinations thereof. Accordingly, in some embodiments, information can be communicated to the ultrasound systems 1102 and 1104 through the network 1106. For instance, the database 1108 can store instructions executable by a processor system of the ultrasound systems 1102 and 1104, and communicate the instructions via the network 1106. Additionally or alternatively, the database 1108 can maintain classification waveforms, such as the classification waveforms 308-324 in FIG. 3, and communicate them to one or both of the ultrasound systems 1102 and 1104 for determining venous congestion with ultrasound.

The environment 1100 can include a medical archiver 1110 (e.g., a vendor neutral archive (VNA), middle ware, EMR, PACS, etc.) to be able to visualize into the patient's history without being limited to the studies on the one ultrasound system the user is currently using that maintains patient medical records. In some embodiments, the use of a VNA, middle ware, EMR, PACS, etc. enables visualization into the patient's history without being limited to the studies on the one ultrasound system the user is currently using. The medical archiver 1110 is coupled to the network 1106, and can be in communication with the ultrasound systems 1102 and 1104 via the network 1106. For instance, the medical archiver 1110 can store medical data (e.g., ultrasound examination data) generated by the ultrasound systems 1102 and 1104, and provide medical data (e.g., data from previous ultrasound examinations) to the ultrasound systems 1102 and 1104 for use in a current ultrasound examination, such as for comparing Doppler waveforms.

The environment 1100 also includes a server system 1112 that can implement any of the functions described herein. The server system 1112 can be a separate device from the ultrasound systems 1102 and 1104. Alternatively, the server system 1112 can be included in at least one of the ultrasound systems 1102 and 1104. In some embodiments, the server system 1112 and the database 1108 are included in at least one of the ultrasound systems 1102 and 1104. In an example, the server system 1112 is implemented as a remote server system that is remote from (e.g., not collocated with) the ultrasound systems 1102 and 1104.

Many of the aspects described herein can be implemented using a machine-learned model. For the purposes of this disclosure, a machine-learned model is any model that accepts an input, analyzes and/or processes the input based on an algorithm derived via machine-learning training, and provides an output. A machine-learned model can be conceptualized as a mathematical function of the following form: f(ŝ, θ)=ŷ Equation (1)

In Equation (1), the operator f represents the processing of the machine-learned model based on an input and providing an output. The term ŝ represents a model input, such as ultrasound data. The model analyzes/processes the input ŝ using parameters θ to generate output ŷ (e.g., object identification, object segmentation, object classification, etc.). Both ŝ and ŷ can be scalar values, matrices, vectors, or mathematical representations of phenomena such as categories, classifications, image characteristics, the images themselves, text, labels, or the like. The parameters θ can be any suitable mathematical operations, including but not limited to applications of weights and biases, filter coefficients, summations or other aggregations of data inputs, distribution parameters such as mean and variance in a Gaussian distribution, linear algebra-based operators, or other parameters, including combinations of different parameters, suitable to map data to a desired output.

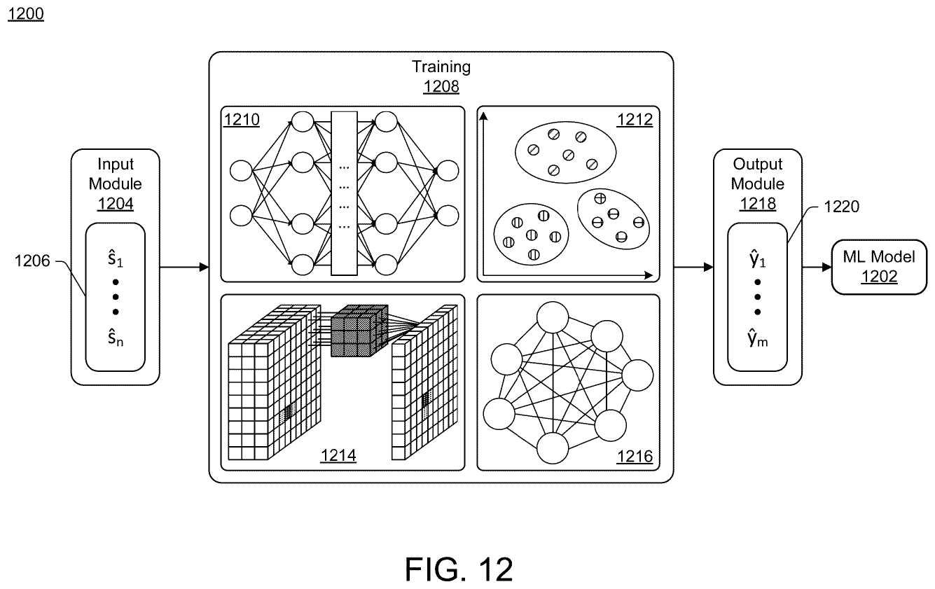

FIG. 12 represents an example machine-learning architecture 1200 used to train a machine-learned model M 1202. An input module 1204 accepts an input ŝ 1206, which can be an array with members ŝ₁ through ŝₙ. The input ŝ 1206 is fed into a training module 1208, which processes the input ŝ 1206 based on the machine-learning architecture 1200. For example, if the machine-learning architecture 1200 uses a multilayer perceptron (MLP) model 1210, the training module 1208 applies weights and biases to the input ŝ 1206 through one or more layers of perceptrons, each perceptron performing a fit using its own weights and biases according to its given functional form. MLP weights and biases can be adjusted so that they are optimized against a least mean square, logcosh, or other optimization function (e.g., loss function) known in the art. Although an MLP model 1210 is described here as an example, any suitable machine-learning technique can be employed, some examples of which include but are not limited to k-means clustering 1212, convolutional neural networks (CNN) 1214, a Boltzmann machine 1216, Gaussian mixture models (GMM), and long short-term memory (LSTM). The training module 1208 provides an input to an output module 1218. The output module 1218 analyzes the input from the training module 1208 and provides an output in the form of ŷ 1220, which can be an array with members ŷ₁ through ŷₘ. The output 1220 can represent a known correlation with the input ŝ 1206, such as, for example, object identification, segmentation, and/or classification.

In some embodiments, the input ŝ 1206 can be a training input labeled with known output correlation values, and these known values can be used to optimize the output ŷ 1220 in training against the optimization/loss function. In some other embodiments, the machine-learning architecture 1200 can categorize the output ŷ 1220 values without being given known correlation values to the inputs ŝ 1206. In some embodiments, the machine-learning architecture 1200 can be a combination of machine-learning architectures. By way of example, a first network can use the input ŝ 1206 and provide the output ŷ 1220 as an input ŝ_{ML} to a second machine-learned architecture, with the second machine-learned architecture providing a final output ŷ_{f}. In another example, one or more machine-learning architectures can be implemented at various points throughout the training module 1208.

In some embodiments of machine-learned models, all layers of the model are fully connected. For example, all perceptrons in an MLP model act on every member of ŝ. For an MLP model with a 100×100 pixel image as the input, each perceptron provides weights/biases for 10,000 inputs. With a large, densely layered model, this may result in slower processing and/or issues with vanishing and/or exploding gradients. A CNN, which may not be a fully connected model, can process the same image using 5×5 tiled regions, requiring only 25 perceptrons with shared weights, giving much greater efficiency than the fully connected MLP model.

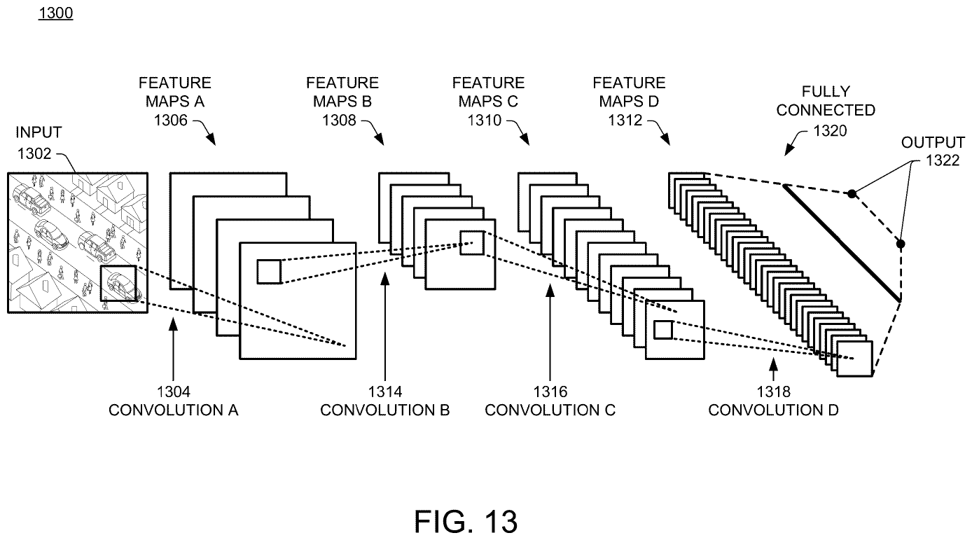

FIG. 13 represents some embodiments of model 1300 using a CNN to process an input image 1302, which includes representations of objects that can be identified via object recognition, such as people or cars (or an anatomy, as described in relation to FIGS. 1-12). Convolution A 1304 can be performed to create a first set of feature maps (e.g., feature maps A 1306). A feature map can be a mapping of aspects of the input image 1302 given by a filter element of the CNN. This process can be repeated using feature maps A 1306 to generate further feature maps B 1308, feature maps C 1310, and feature maps D 1312 using convolution B 1314, convolution C 1316, and convolution D 1318, respectively. In some embodiments, the feature maps D 1312 become an input for fully connected network layers 1320. In this way, the machine-learned model can be trained to recognize certain elements of the image, such as people, cars, or a particular patient anatomy, and provide an output 1322 that, for example, identifies the recognized elements. In some embodiments, an inference generated with an ultrasound system can be appended to a feature map (e.g., feature map B 1308) generated by a neural network (e.g., CNN). In this way, the feature vector and/or inference can be used as a secondary/conditional input to the neural network.

Although the example of FIG. 13 shows a CNN as a part of a fully connected network, other architectures are possible and this example should not be seen as limiting. There can be more or fewer layers in the CNN. A CNN component for a model can be placed in a different order, or the model can contain additional components or models. There may be no fully connected components, such as a fully convolutional network. Additional aspects of the CNN, such as pooling, downsampling, upsampling, or other aspects known to people skilled in the art can also be employed.

### Example Procedures

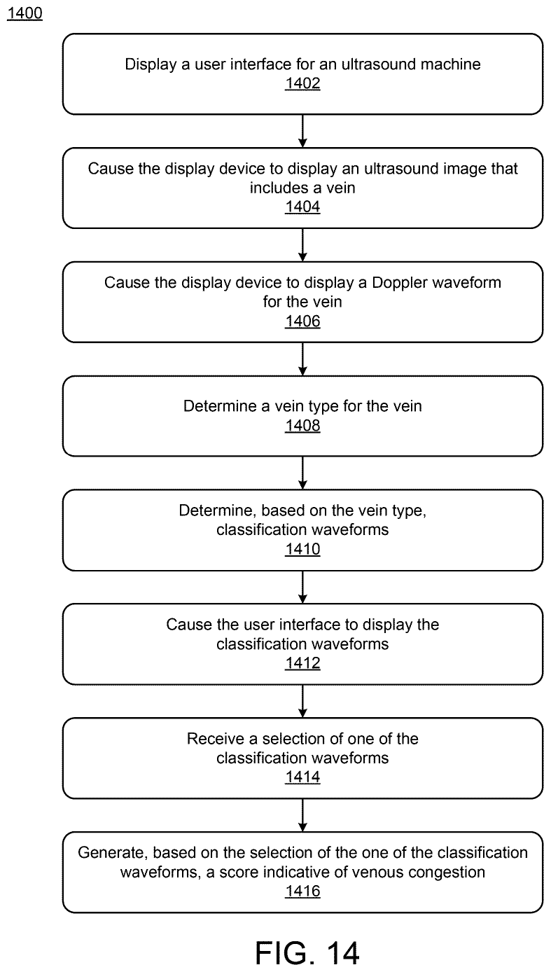

FIG. 14 illustrates some embodiments of method 1400 that can be implemented by an ultrasound system (e.g., the ultrasound system 900 of FIG. 9) for determining venous congestion with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 10.

Referring to FIG. 14, a user interface for an ultrasound machine is displayed (block 1402). The user interface can be displayed on a clinical display of a display device that is part of an ultrasound machine. In some embodiments, the user interface is displayed by a display device that is coupled to the ultrasound machine, such as a tablet. The display device is caused to display an ultrasound image that includes a vein (block 1404). The display device is caused to display a Doppler waveform for the vein (block 1406). A vein type for the vein is determined (block 1408). Based on the vein type, classification waveforms are determined (block 1410). The user interface is caused to display the classification waveforms (block 1412). A selection of one of the classification waveforms is received (block 1414). Based on the selection of the one of the classification waveforms, a score indicative of venous congestion is generated (block 1416).

In some embodiments, the selection is received as a user selection via the user interface. For instance, the user selection can include a touch input on the user interface for the selection of the one of the classification waveforms. As an example, a user can touch a visual representation of the one of the classification waveforms.

In some embodiments, when determining venous congestion with ultrasound, a processor system implements a machine-learned model to generate, based on the Doppler waveform for the vein, the selection of the one of the classification waveforms. For example, the machine-learned model can determine the one of the classification waveforms based on a degree of matching of a Doppler waveform to the classification waveforms, e.g., a best fit, such as according to minimizing a mean-squared error between the Doppler waveform and the classification waveforms. The machine-learned model can generate the degree of matching, and the user interface can display the degree of matching.

In some embodiments, the ultrasound system includes a transmitter, and the processor system is implemented to populate a medical worksheet with the score. The transmitter can communicate the medical worksheet to a medical archiver, and the display device can display the medical worksheet.

In some embodiments, the processor system causes the display device to display an additional ultrasound image that includes an additional vein. The processor system can cause the display device to display an additional Doppler waveform for the additional vein, and determine an additional vein type for the additional vein. Based on the additional vein type, the processor system can determine additional classification waveforms. The processor system can cause the user interface to display the additional classification waveforms. The processor system can receive an additional selection of one of the additional classification waveforms, and update, based on the additional selection of the one of the additional classification waveforms, the score indicative of venous congestion. The processor system can then cause the display device to display the updated score.

In some embodiments, the display device displays a range of possible scores and indicates the score on the range. For instance, the range can be displayed as an interval, and the score with a hash mark on the interval.

In some embodiments, the vein type is selected from the group consisting of a hepatic vein, a portal vein, and a renal vein, wherein the classification waveforms indicate degrees of abnormality. The user interface can simultaneously display the classification waveforms for the hepatic vein, the portal vein, and the renal vein. The processor can cause the user interface to emphasize the display of the classification waveforms for the vein type. The emphasis can enlarge the classification waveforms for the vein type compared to the classification waveforms for the other vein types, brighten the classification waveforms for the vein type, grey out or make partially opaque the classification waveforms for the other vein types, and the like.

In some embodiments, the user interface displays the classification waveforms at a first location of the user interface. The processor system determines an additional vein type for an additional vein included in an additional ultrasound image, and determines, based on the additional vein type, additional classification waveforms. The processor system can then cause the display device to remove the classification waveforms displayed at the first location and display the additional classification waveforms at the first location.

In some embodiments, when determining venous congestions with ultrasound, the processor system determines the vein type based on a current step of an ultrasound protocol implemented by the ultrasound machine. For instance, based on a user selection of the current step of the ultrasound protocol, such as a “hepatic vein” step of a VExUS protocol, the processor system can determine the vein type (e.g., “hepatic vein”).

In some embodiments, the processor system generates the Doppler waveform for the vein as part of a current ultrasound examination for a patient, and obtains, from a memory device, additional Doppler waveforms for the vein obtained as part of one or more previous ultrasound examinations for the patient. The processor system can cause the display device to display a visual representation that indicates the one or more previous ultrasound examinations, and cause the display device to display the additional Doppler waveforms for a previous ultrasound examination of the one or more previous ultrasound examinations responsive to a user selection of the previous ultrasound examination from the visual representation. For instance, the display device can display a time line of the previous ultrasound examinations, and when a user selects one of the previous ultrasound examinations, the processor system can cause the display device to display additional Doppler waveforms from the one of the previous ultrasound examinations. The processor system can retrieve the additional Doppler waveforms from a medical archiver or middleware, and not solely the system the user is on.

In some embodiments, the user interface receives a user trace of the Doppler waveform via touch input. The processor system can compare the user trace of the Doppler waveform to the classification waveforms, and determine, based on the comparison, a selection of one of the classification waveforms. The processor system can generate, based on the selection of the one of the classification waveforms, the score indicative of venous congestion.

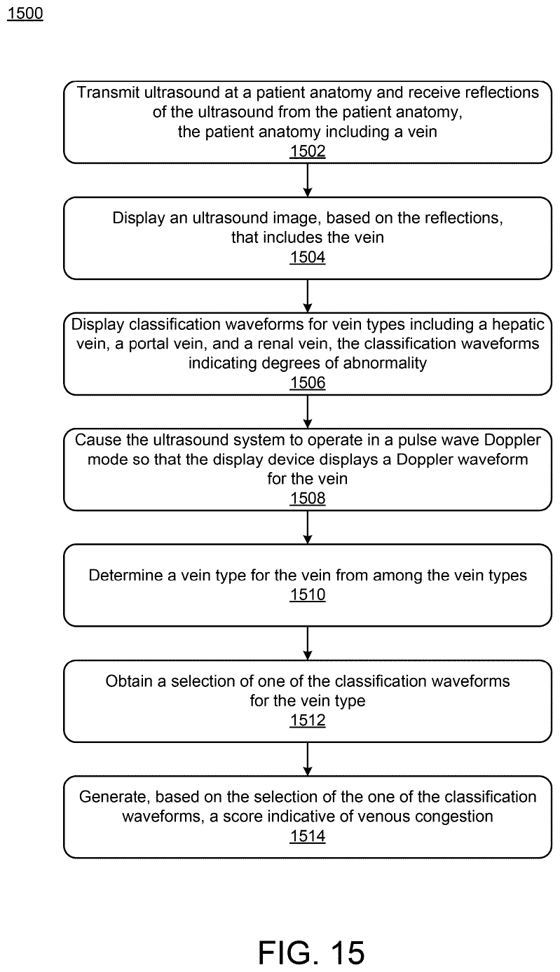

FIG. 15 illustrates some embodiments of method 1500 that can be implemented by an ultrasound system (e.g., the ultrasound system 900 of FIG. 9) in accordance with the present invention for determining venous congestion with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 10.

Referring to FIG. 15, ultrasound is transmitted at a patient anatomy and reflections of the ultrasound from the patient anatomy are received, the patient anatomy including a vein (block 1502). An ultrasound image, based on the reflections, that includes the vein, is displayed (block 1504). Classification waveforms for vein types including a hepatic vein, a portal vein, and a renal vein are displayed, the classification waveforms indicating degrees of abnormality (block 1506). The ultrasound system is caused to operate in a pulse wave Doppler mode so that the display device displays a Doppler waveform for the vein (block 1508). A vein type for the vein is determined from among the vein types (block 1510). A selection of one of the classification waveforms for the vein type is obtained (block 1512). Based on the selection of the one of the classification waveforms, a score indicative of venous congestion is generated (block 1514).

In some embodiments, the processor system obtains a selection of a second one of the classification waveforms for a second vein type from among the vein types for a second vein. The processor system can generate the score based on the selection of the second one of the classification waveforms. In an example, the processor system obtains a selection of a third one of the classification waveforms for a third vein type from among the vein types for a third vein, and updates the score based on the selection of the third one of the classification waveforms. The display device can refresh a display of the score based on the update. The refresh can include to remove the score before the update, and replace it with the updated score.

In some embodiments, the system takes multiple waveforms (e.g., three waveforms) from the same spectral trace dataset and averages them out to obtain the best guess. For example, the data can be collected from one location, three waveforms can be obtained to analyze at that same location, and then the system averages them out to find the best fit for normal, mild, moderate, severe. This averaging can happen at each doppler location (or 2D location) that is done. In this case, the hepatic vein would have one averaged classification.

In some embodiments, the display device can automatically adjust a scale of the display for the Doppler waveform based on the determination of the vein type. The scale can include one or more of a time scale, a gain scale, a frequency scale, and a resolution.

In some embodiments, the processor system generates a recommendation based on the score. The recommendation can include at least one of a change in fluid status, a time frame for a subsequent examination, and a predicted result for the subsequent examination. The predicted result can include an expected score for venous congestion and/or a degree of abnormality of a Doppler waveform for at least one of a hepatic vein, a portal vein, and a renal vein.

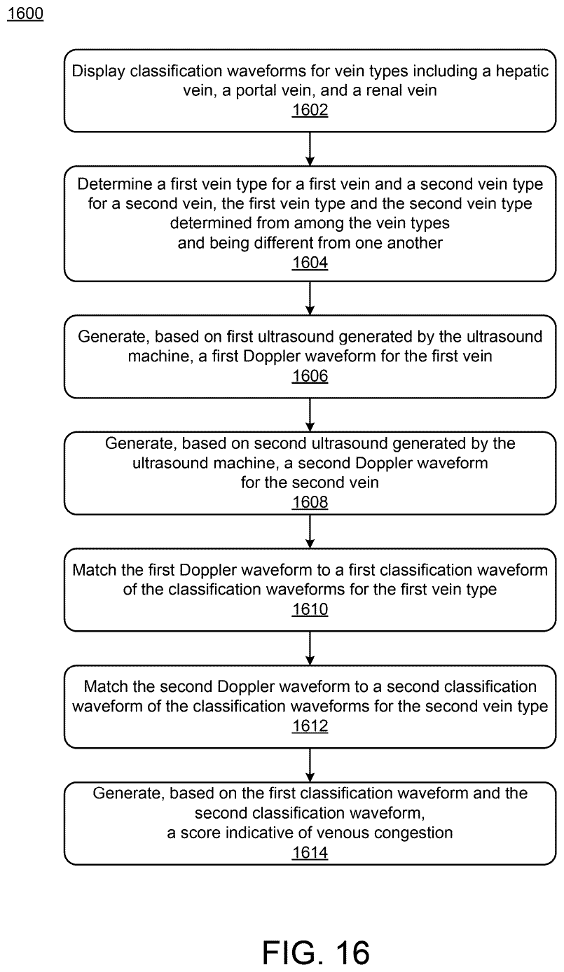

FIG. 16 illustrates some embodiments of method 1600 that can be implemented by an ultrasound system (e.g., the ultrasound system 900 of FIG. 9) for determining venous congestion with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 10.

Referring to FIG. 16, classification waveforms for vein types including a hepatic vein, a portal vein, and a renal vein are displayed (block 1602). A first vein type for a first vein and a second vein type for a second vein are determined, the first vein type and the second vein type determined from among the vein types and being different from one another (block 1604). Based on first ultrasound generated by the ultrasound machine, a first Doppler waveform for the first vein is generated (block 1606). Based on second ultrasound generated by the ultrasound machine, a second Doppler waveform for the second vein is generated (block 1608). The first Doppler waveform is matched to a first classification waveform of the classification waveforms for the first vein type (block 1610). The second Doppler waveform is matched to a second classification waveform of the classification waveforms for the second vein type (block 1612). Based on the first classification waveform and the second classification waveform, a score indicative of venous congestion is generated (block 1614).

In some embodiments, the display device displays a measure of the match of the first Doppler waveform to the first classification waveform and a measure of the match of the second Doppler waveform to the second classification waveform. For instance, the measures can include a percent error between the Doppler waveforms and the first and second classification waveforms. Additionally or alternatively, a measure of the match between a Doppler waveform and a classification waveform can be determined by the integral of the magnitude of the difference between a Doppler waveform and a classification waveform. Additionally or alternatively, a measure of the match between a Doppler waveform and a classification waveform can be determined by the integral of the magnitude squared of the difference between a Doppler waveform and a classification waveform. The integral can be normalized in any suitable way, to place the measure in a desired range, such as between zero and one.

In some embodiments, the display device displays the classification waveforms simultaneously with an ultrasound image that includes at least one of the first vein and the second vein. Additionally or alternatively, the processor system can automatically and without user intervention place a pulse wave Doppler gate on the ultrasound image, the pulse wave Doppler gate used to generate the first Doppler waveform or the second Doppler waveform. The processor system can implement a machine-learned model to generate the placement of the Doppler gate, e.g., based on the ultrasound image and the vein type.

In some embodiments, the ultrasound system includes a transesophageal ultrasound scanner implemented to generate at least one of the first ultrasound and the second ultrasound. The scanner 104-2 in FIG. 2 is an example of a transesophageal ultrasound scanner. The user interface of the ultrasound system can include a control option to indicate to the ultrasound system that a transesophageal ultrasound scanner is enabled for determining venous congestion with ultrasound. Responsive to the enablement of the transesophageal ultrasound scanner, the ultrasound system can obtain visual representation for such use, compared to a conventional hand-held scanner for external use, such as the scanner 104-1 in FIG. 2. The visual representations can include representations (e.g., icons) for probe guidance and placement, such as those included in the panels 412, 512, and 612 in FIGS. 4-6, respectively. In embodiments, the visual representations include example views of the imaging plane in an ultrasound image, to guide the user to hold the probe to match the example views. The database 1108 in FIG. 11 can provide the visual representations to the ultrasound system, which can be stored by an ultrasound machine (e.g., the ultrasound machine 102).

In some embodiments, the processor system generates a pulsatility fraction for at least one of the first Doppler waveform and the second Doppler waveform. At least one of the match of the first Doppler waveform to the first classification waveform or the match of the second Doppler waveform to the second classification waveform can based on the pulsatility fraction. For instance, the ultrasound system can select a classification waveform that has a closest average pulsatility fraction to the pulsatility fraction calculated for a Doppler waveform.

In embodiments, the display device displays an indicator that a sufficient quantity of data has been collected to generate the score. For example, the display device can display text as the indicator, such as the text “Sufficient Data Available for Venous Score”. Additionally or alternatively, the indicator can include an icon (e.g., a fill gauge, like a gas gauge), a gif, an animation, a thumbnail image, etc.

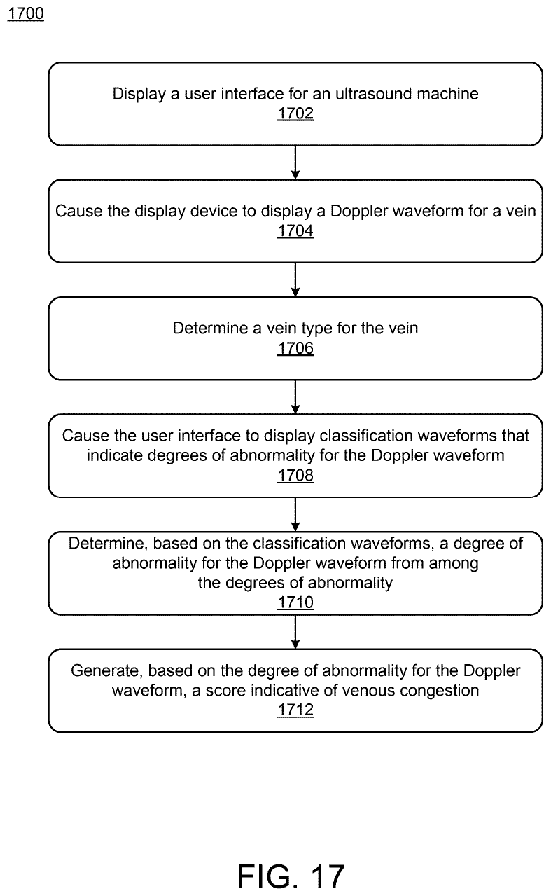

FIG. 17 illustrates some embodiments of method 1700 that can be implemented by an ultrasound system (e.g., the ultrasound system 900 of FIG. 9) for determining venous congestion with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 10.

A user interface for the ultrasound machine is displayed (block 1702). The display device is caused to display a Doppler waveform for a vein (block 1704). A vein type is determined for the vein (block 1706). The user interface is caused to display classification waveforms that indicate degrees of abnormality for the Doppler waveform (block 1708). Based on the classification waveforms, a degree of abnormality for the Doppler waveform from among the degrees of abnormality is determined (block 1710). Based on the degree of abnormality for the Doppler waveform, a score indicative of venous congestion is generated (block 1712).

In some embodiments, the degree of abnormality is selected from the group consisting of normal, mildly abnormal, and severely abnormal. Additionally or alternatively, the score can be constrained to integer values between and including zero to three.

In some embodiments of determining venous congestion with ultrasound, the processor system can, based on additional classification waveforms for an additional vein type for an additional vein, determine an additional degree of abnormality for an additional Doppler waveform for the additional vein. The generation of the score can be based on the additional degree of abnormality. Additionally, the processor system can, based on third classification waveforms for a third vein type for a third vein, determine a third degree of abnormality for a third Doppler waveform for the third vein. The generation of the score can be based on the third degree of abnormality. In an example, the processor system determines at least one of the degree of abnormality for the Doppler waveform, the additional degree of abnormality for the additional Doppler waveform, and the third degree of abnormality for the third Doppler waveform, based on a user trace of at least one of the Doppler waveform, the additional Doppler waveform, and the third Doppler waveform.

In some embodiments, the processor system implements a machine-learned model to determine the vein type for the vein. The processor system can implement the machine-learned model to distinguish the vein from an artery before the determination of the vein type for the vein.

In embodiments, the user interface is implemented to display the classification waveforms simultaneously with additional classification waveforms for an additional vein type. In other embodiments, the user interface is implemented to display the classification waveforms for the vein type without simultaneous display of additional classification waveforms for an additional vein type.

In aspects, the processor system can determine, based on the vein type, a time scale, and cause the display device to display the Doppler waveform in the time scale. The display of the Doppler waveform in the time scale can include to change the display of the Doppler waveform from an additional time scale to the time scale.

The systems, devices, and procedures disclosed herein constitute numerous advantages over conventional systems, devices, and procedures that merely provide a printed VExUS scorecard, or a scorecard that is downloaded to a personal device (e.g., a smartphone). In contrast, the systems, devices, and procedures disclosed herein exemplify “ease of use” and simplified workflow, by saving time and resources and allowing the user to focus on the clinical display when determining venous congestion with ultrasound. Moreover, the systems, devices, and procedures disclosed herein reduce ambiguities, user-dependent interpretation of results, and errors resulting from poor memory recall by a user, as are common with conventional systems, devices, and procedures. By removing the hard-copy or downloaded VExUS scorecard associated with conventional systems, devices, and procedures, the systems, devices, and procedures disclosed herein also remove the sanitary issues associated with the hard-copy or downloaded VExUS scorecard. Further, the VExUS information is always available during an ultrasound examination and never lost, unlike with conventional systems, devices, and procedures. Accordingly, by using the systems, devices, and procedures disclosed herein instead of conventional systems, devices, and procedures, patients receive better care. Moreover, having the VExUS algorithm embedded in the ultrasound ensures all practitioners are referring to the same clinical resource and can deliver the same standard of care.

Note that the techniques disclosed herein for waveform matching to generate a score can be used in other applications. For example, the techniques can be used for downstream stenosis-where there is a waveform pattern that suggests a stenosis downstream. This is referred to as blunting.

There are a number of example embodiments described herein.

Example 1 is an ultrasound machine including a display device configured to display a user interface for the ultrasound machine and a processor system. The processor system is configured to cause the display device to display an ultrasound image that includes a vein, cause the display device to display a Doppler waveform for the vein, determine a vein type for the vein, determine classification waveforms based on the vein type, and cause the user interface to display the classification waveforms.

Example 2 is the ultrasound machine of example 1 that may optionally include that the processor system is implemented to receive a selection of one of the classification waveforms; and generate, based on the selection of the one of the classification waveforms, a score indicative of venous congestion.

Example 3 is the ultrasound machine of example 2 that may optionally include that the processor system is implemented to receive the selection as a user selection via the user interface.

Example 4 is the ultrasound machine of example 3 that may optionally include that the user selection includes a touch input on the user interface for the selection of the one of the classification waveforms.

Example 5 is the ultrasound machine of example 2 that may optionally include that the processor system is configured to implement a machine-learned model to generate, based on the Doppler waveform for the vein, the selection of the one of the classification waveforms.

Example 6 is the ultrasound machine of example 2 that may optionally include a transmitter, wherein the processor system is implemented to populate a medical worksheet with the score, wherein the transmitter is implemented to communicate the medical worksheet to a medical archiver, wherein the display device is implemented to display the medical worksheet.

Example 7 is the ultrasound machine of example 2 that may optionally include that the processor system is implemented to cause the display device to display an additional ultrasound image that includes an additional vein, cause the display device to display an additional Doppler waveform for the additional vein, and determine an additional vein type for the additional vein. The processor system is also implemented to determine additional classification waveform based on the additional vein types, cause the user interface to display the additional classification waveforms, receive an additional selection of one of the additional classification waveforms, update the score indicative of venous congestion based on the additional selection of the one of the additional classification waveforms, and cause the display device to display the updated score.

Example 8 is the ultrasound machine of example 2 that may optionally include that the display device is implemented to display a range of possible scores and indicate the score on the range.

Example 9 is the ultrasound machine of example 1 that may optionally include that the vein type is selected from the group consisting of a hepatic vein, a portal vein, and a renal vein, wherein the classification waveforms indicate degrees of abnormality.

Example 10 is the ultrasound machine of example 9 that may optionally include that the user interface is implemented to simultaneously display the classification waveforms for the hepatic vein, the portal vein, and the renal vein, wherein the processor is implemented to cause the user interface to emphasize the display of the classification waveforms for the vein type.

Example 11 is the ultrasound machine of example 1 that may optionally include that the user interface is implemented to display the classification waveforms at a first location of the user interface, wherein the processor system is implemented to determine an additional vein type for an additional vein included in an additional ultrasound image determine, based on the additional vein type, additional classification waveforms, and cause the display device to remove the classification waveforms displayed at the first location and display the additional classification waveforms at the first location.

Example 12 is the ultrasound machine of example 1 that may optionally include that the processor system is implemented to determine the vein type based on a current step of an ultrasound protocol implemented by the ultrasound machine.

Example 13 is the ultrasound machine of example 1 that may optionally include that the processor system is implemented to generate the Doppler waveform for the vein as part of a current ultrasound examination for a patient, obtain, from a memory device, additional Doppler waveforms for the vein obtained as part of one or more previous ultrasound examinations for the patient, cause the display device to display a visual representation that indicates the one or more previous ultrasound examinations, and cause the display device to display the additional Doppler waveforms for a previous ultrasound examination of the one or more previous ultrasound examinations responsive to a user selection of the previous ultrasound examination from the visual representation.

Example 14 is the ultrasound machine of example 1 that may optionally include that the user interface is implemented to receive a user trace of the Doppler waveform via touch input, wherein the processor system is implemented to compare the user trace of the Doppler waveform to the classification waveforms, determine a selection of one of the classification waveforms based on the comparison, and generate a score indicative of venous congestion based on the selection of the one of the classification waveforms.

Example 15 is an ultrasound system including: an ultrasound scanner configured to transmit ultrasound at a patient anatomy and receive reflections of the ultrasound from the patient anatomy, the patient anatomy including a vein, and a display device. The display device is configured to display an ultrasound image, based on the reflections, that includes the vein, and classification waveforms for vein types including a hepatic vein, a portal vein, and a renal vein, the classification waveforms indicating degrees of abnormality. The ultrasound system also includes a processor system configured to cause the ultrasound system to operate in a pulse wave Doppler mode so that the display device displays a Doppler waveform for the vein, determine a vein type for the vein from among the vein types, obtain a selection of one of the classification waveforms for the vein type, and generate, based on the selection of the one of the classification waveforms, a score indicative of venous congestion.

Example 16 is the ultrasound system of example 15 that may optionally include that the processor system is implemented to obtain a selection of a second one of the classification waveforms for a second vein type from among the vein types for a second vein, wherein the generation of the score is based on the selection of the second one of the classification waveforms.

Example 17 is the ultrasound system of example 16 that may optionally include that the processor system is implemented to obtain a selection of a third one of the classification waveforms for a third vein type from among the vein types for a third vein and update the score based on the selection of the third one of the classification waveforms, wherein the display device is implemented to refresh a display of the score based on the update.

Example 18 is the ultrasound system of example 15 that may optionally include that the display device is implemented to automatically adjust a scale of the display for the Doppler waveform based on the determination of the vein type.

Example 19 is the ultrasound system of example 15 that may optionally include that the processor system is implemented to generate a recommendation based on the score, the recommendation including at least one of a change in fluid status, a time frame for a subsequent examination, and a predicted result for the subsequent examination.

Example 20 is ultrasound machine including a display device configured to display classification waveforms for vein types including a hepatic vein, a portal vein, and a renal vein, and a processor system. The processor system is configured to determine a first vein type for a first vein and a second vein type for a second vein, where the first vein type and the second vein type are determined from among the vein types and being different from one another, generate a first Doppler waveform for the first vein based on first ultrasound generated by the ultrasound machine, and generate a second Doppler waveform for the second vein based on second ultrasound generated by the ultrasound machine. The processor system is also configured to match the first Doppler waveform to a first classification waveform of the classification waveforms for the first vein type, match the second Doppler waveform to a second classification waveform of the classification waveforms for the second vein type, and generate a score indicative of venous congestion based on the first classification waveform and the second classification waveform.

Example 21 is the ultrasound machine of example 20 that may optionally include that the display device is implemented to display a measure of the match of the first Doppler waveform to the first classification waveform and a measure of the match of the second Doppler waveform to the second classification waveform.

Example 22 is the ultrasound machine of example 20 that may optionally include that the display device is implemented to display the classification waveforms simultaneously with an ultrasound image that includes at least one of the first vein and the second vein.

Example 23 is the ultrasound machine of example 22 that may optionally include that the processor system is implemented to automatically and without user intervention place a pulse wave Doppler gate on the ultrasound image, the pulse wave Doppler gate used to generate the first Doppler waveform or the second Doppler waveform.

Example 24 is the ultrasound machine of example 20 that may optionally include a transesophageal ultrasound probe implemented to generate at least one of the first ultrasound and the second ultrasound.

Example 25 is the ultrasound machine of example 20 that may optionally include that the processor system is implemented to generate a pulsatility fraction for at least one of the first Doppler waveform and the second Doppler waveform, and at least one of the match of the first Doppler waveform to the first classification waveform or the match of the second Doppler waveform to the second classification waveform is based on the pulsatility fraction.

Example 26 is the ultrasound machine of example 20 that may optionally include that the display device is implemented to display an indicator that a sufficient quantity of data has been collected to generate the score.

Example 27 is an ultrasound machine including a display device configured to display a user interface for the ultrasound machine and a processor system. The processor system is configured to cause the display device to display a Doppler waveform for a vein, determine a vein type for the vein, and cause the user interface to display classification waveforms that indicate degrees of abnormality for the Doppler waveform. The processor system is also configured to determine a degree of abnormality for the Doppler waveform from among the degrees of abnormality based on the classification waveforms and generate a score indicative of venous congestion based on the degree of abnormality for the Doppler waveform.

Example 28 is the ultrasound machine of example 27 that may optionally include that the degree of abnormality is selected from the group consisting of normal, mildly abnormal, and severely abnormal.

Example 29 is the ultrasound machine of example 27 that may optionally include that the processor system is implemented to, based on additional classification waveforms for an additional vein type for an additional vein, determine an additional degree of abnormality for an additional Doppler waveform for the additional vein, wherein the generation of the score is based on the additional degree of abnormality.

Example 30 is the ultrasound machine of example 29 that may optionally include that the processor system is implemented to, based on third classification waveforms for a third vein type for a third vein, determine a third degree of abnormality for a third Doppler waveform for the third vein, wherein the generation of the score is based on the third degree of abnormality.

Example 31 is the ultrasound machine of example 30 that may optionally include that the processor system is implemented to determine at least one of the degree of abnormality for the Doppler waveform, the additional degree of abnormality for the additional Doppler waveform, and the third degree of abnormality for the third Doppler waveform, based on a user trace of at least one of the Doppler waveform, the additional Doppler waveform, and the third Doppler waveform.

Example 32 is the ultrasound machine of example 27 that may optionally include that the processor system is configured to implement a machine-learned model to determine the vein type for the vein.

Example 33 is the ultrasound machine of example 27 that may optionally include that the processor system is configured to implement the machine-learned model to distinguish the vein from an artery before the determination of the vein type for the vein.

Example 34 is the ultrasound machine of example 27 that may optionally include that the user interface is implemented to display the classification waveforms simultaneously with additional classification waveforms for an additional vein type.

Example 35 is the ultrasound machine of example 27 that may optionally include that the user interface is implemented to display the classification waveforms for the vein type without simultaneous display of additional classification waveforms for an additional vein type.

Example 36 is the ultrasound machine of example 27 that may optionally include that the processor system is implemented to determine a time scale based on the vein type and cause the display device to display the Doppler waveform in the time scale.

Example 37 is the ultrasound machine of example 36 that may optionally include that display of the Doppler waveform in the time scale includes to change the display of the Doppler waveform from an additional time scale to the time scale.

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in some embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

We claim:

1. An ultrasound machine comprising:
a display device configured to display a user interface for the ultrasound machine; and
a processor system configured to:
cause the display device to display an ultrasound image that includes a vein;
cause the display device to display a Doppler waveform for the vein;
determine a vein type for the vein;
determine, based on the vein type, classification waveforms; and
cause the user interface to display the classification waveforms.

2. The ultrasound machine as described in claim 1, wherein the processor system is implemented to:
receive a selection of one of the classification waveforms; and
generate, based on the selection of the one of the classification waveforms, a score indicative of venous congestion.

3. The ultrasound machine as described in claim 2, wherein the processor system is implemented to receive the selection as a user selection via the user interface.

4. The ultrasound machine as described in claim 3, wherein the user selection includes a touch input on the user interface for the selection of the one of the classification waveforms.

5. The ultrasound machine as described in claim 2, wherein the processor system is configured to implement a machine-learned model to generate, based on the Doppler waveform for the vein, the selection of the one of the classification waveforms.

6. The ultrasound machine as described in claim 2, further comprising a transmitter, wherein the processor system is implemented to populate a medical worksheet with the score, wherein the transmitter is implemented to communicate the medical worksheet to a medical archiver, wherein the display device is implemented to display the medical worksheet.

7. The ultrasound machine as described in claim 2, wherein the processor system is implemented to:
cause the display device to display an additional ultrasound image that includes an additional vein;
cause the display device to display an additional Doppler waveform for the additional vein;
determine an additional vein type for the additional vein;
determine, based on the additional vein type, additional classification waveforms;
cause the user interface to display the additional classification waveforms;
receive an additional selection of one of the additional classification waveforms;
update, based on the additional selection of the one of the additional classification waveforms, the score indicative of venous congestion; and
cause the display device to display the updated score.

8. The ultrasound machine as described in claim 2, wherein the display device is implemented to display a range of possible scores and indicate the score on the range.

9. The ultrasound machine as described in claim 1, wherein the vein type is selected from the group consisting of a hepatic vein, a portal vein, and a renal vein, wherein the classification waveforms indicate degrees of abnormality.

10. The ultrasound machine as described in claim 9, wherein the user interface is implemented to simultaneously display the classification waveforms for the hepatic vein, the portal vein, and the renal vein, wherein the processor is implemented to cause the user interface to emphasize the display of the classification waveforms for the vein type.

11. The ultrasound machine as described in claim 1, wherein the user interface is implemented to display the classification waveforms at a first location of the user interface, wherein the processor system is implemented to:
determine an additional vein type for an additional vein included in an additional ultrasound image;
determine, based on the additional vein type, additional classification waveforms; and
cause the display device to remove the classification waveforms displayed at the first location and display the additional classification waveforms at the first location.

12. The ultrasound machine as described in claim 1, wherein the processor system is implemented to determine the vein type based on a current step of an ultrasound protocol implemented by the ultrasound machine.

13. The ultrasound machine as described in claim 1, wherein the processor system is implemented to:
generate the Doppler waveform for the vein as part of a current ultrasound examination for a patient;
obtain, from a memory device, additional Doppler waveforms for the vein obtained as part of one or more previous ultrasound examinations for the patient;
cause the display device to display a visual representation that indicates the one or more previous ultrasound examinations; and
cause the display device to display the additional Doppler waveforms for a previous ultrasound examination of the one or more previous ultrasound examinations responsive to a user selection of the previous ultrasound examination from the visual representation.

14. The ultrasound machine as described in claim 1, wherein the user interface is implemented to receive a user trace of the Doppler waveform via touch input, wherein the processor system is implemented to:
compare the user trace of the Doppler waveform to the classification waveforms;
determine, based on the comparison, a selection of one of the classification waveforms; and
generate, based on the selection of the one of the classification waveforms, a score indicative of venous congestion.

15. An ultrasound system comprising:
an ultrasound scanner configured to transmit ultrasound at a patient anatomy and receive reflections of the ultrasound from the patient anatomy, the patient anatomy including a vein;
a display device configured to display:
an ultrasound image, based on the reflections, that includes the vein; and
classification waveforms for vein types including a hepatic vein, a portal vein, and a renal vein, the classification waveforms indicating degrees of abnormality; and
a processor system configured to:
cause the ultrasound system to operate in a pulse wave Doppler mode so that the display device displays a Doppler waveform for the vein;
determine a vein type for the vein from among the vein types;
obtain a selection of one of the classification waveforms for the vein type; and
generate, based on the selection of the one of the classification waveforms, a score indicative of venous congestion.

16. The ultrasound system as described in claim 15, wherein the processor system is implemented to obtain a selection of a second one of the classification waveforms for a second vein type from among the vein types for a second vein, wherein the generation of the score is based on the selection of the second one of the classification waveforms.

17. The ultrasound system as described in claim 16, wherein the processor system is implemented to:
obtain a selection of a third one of the classification waveforms for a third vein type from among the vein types for a third vein; and
update the score based on the selection of the third one of the classification waveforms, wherein the display device is implemented to refresh a display of the score based on the update.

18. The ultrasound system as described in claim 15, wherein the display device is implemented to automatically adjust a scale of the display for the Doppler waveform based on the determination of the vein type.

19. The ultrasound system as described in claim 15, wherein the processor system is implemented to generate a recommendation based on the score, the recommendation including at least one of a change in fluid status, a time frame for a subsequent examination, and a predicted result for the subsequent examination.

20. An ultrasound machine comprising:
a display device configured to display classification waveforms for vein types including a hepatic vein, a portal vein, and a renal vein; and
a processor system configured to:
determine a first vein type for a first vein and a second vein type for a second vein, the first vein type and the second vein type determined from among the vein types and being different from one another;
generate, based on first ultrasound generated by the ultrasound machine, a first Doppler waveform for the first vein;
generate, based on second ultrasound generated by the ultrasound machine, a second Doppler waveform for the second vein;
match the first Doppler waveform to a first classification waveform of the classification waveforms for the first vein type;
match the second Doppler waveform to a second classification waveform of the classification waveforms for the second vein type; and
generate, based on the first classification waveform and the second classification waveform, a score indicative of venous congestion.
